SEQUENCE LISTING (1 sequence)

SEQ ID NO: 1 - protein, 232 aa, Artificial Sequence
  Description of Artificial Sequence Synthetic polypeptide
MSAYSNTYQEFTNIDQAKAWGNAQYKKYGLSKSEKEAIVSYTKSASEINGKLRQNKGVINGFPSNLIKQVELLDKSFNKMKTPENIMLFRGDDPAYLGTEFQNTLLNSNGTINKTAFEKAKAKFLNKDRLEYGYISTSLMNVSQFAGRPIITKFKVAKGSKAGYIDPISAFAGQLEMLLPRHSTYHIDDMRLSSDGKQIIITATMMGTAINPKEFVMNPANAQGRHTPGTRL

(19) United States
(12) Patent Application Publication
(10) Pub. No.: US 2009/0252712 A1
(43) Pub. Date: Oct. 8, 2009

(54) CHIMERIC C3-LIKE RHO ANTAGONIST BONE THERAPEUTIC

(75) Inventors: Noel James Cusack, Hopkinton, MA (US); Jon Scott Munzer, Franklin, MA (US)

Correspondence Address: WILMERHALE/BOSTON, 60 STATE STREET, BOSTON, MA, 02109, US

(73) Assignee: Alseres Pharmaceuticals, Inc., Hopkington, MA (US)

(21) Appl. No.: 12/400,403

(22) Filed: Mar. 9, 2009

Related U.S. Application Data
(60) Provisional application No. 61/034,668, filed on Mar. 7, 2008.

(51) Int. Cl. A61K 45/00; A61K 38/16; C12N 5/06; A61P 19/00

(52) U.S. Cl. 424/93.7; 435/375; 514/12; 435/377

(57) ABSTRACT

Methods of treating bone disorders in a subject in need thereof by administering to said subject a therapeutically effective amount of a Rho antagonist are disclosed.

### CROSS REFERENCE TO RELATED APPLICATIONS

This application claims the benefit of U.S. Provisional Application No. 61/034,668, filed Mar. 7, 2008, the entire contents of which are hereby incorporated by reference herein.

### FIELD OF THE INVENTION

This invention is in the field of medicine and cell biology. More specifically, the invention relates to treatment of bone cells and bone disorders such as bone fractures and osteoporosis with Rho antagonists.

### BACKGROUND OF THE INVENTION

The maintenance of the mechanical integrity of the skeleton depends on bone remodeling. The main cells involved in bone homeostasis are osteoblasts, osteoclasts, and osteocytes, which are under the control of a host of cytokines released from bone cells and other cells and tissues. Skeletal maintenance requires a well-coordinated balance between bone formation by osteoblasts and bone resorption by osteoclasts.

Osteoclasts are primary bone resorbing cells that play a critical role in bone remodeling. Stimulated osteoblasts transform osteoclast precursor cells into osteoclasts, which fuse together to form giant osteoclasts that dissolve bone. Osteoclasts adhere to the bone surface through specialized discrete structures called “podosomes”, which are composed of F-actin and other cytoskeletal proteins. The formation of actin rings in osteoclasts precedes the initiation of bone resorption, and the ringed structure of podosomes appears at the peripheral region which corresponds exactly to the clear zone of bone-resorbing.

The coupled action of osteoblasts and osteoclasts is regulated by the action of many local and circulating hormones and factors. If uncoupling of these actions occurs, such that bone resorption exceeds bone formation, bone loss, leading to osteoporosis and fragility fractures may result.

A variety of pathological conditions are characterized by the need for enhanced bone formation that occurs as a result of trauma, where sufficient osteogenic activity is crucial for proper and complete restoration of the damaged bone structure. Although therapies for the prevention of osteoporosis exist, such therapies have a limited ability to improve or enhance bone formation. There is great interest, therefore, in agents that positively affect bone mass by stimulating bone formation.

### SUMMARY OF THE INVENTION

In one aspect, the invention features a method of modulating a Rho protein in a bone cell. The method includes contacting the bone cell with a Rho antagonist that modulates a Rho protein level of expression or activity.

In some embodiments, the Rho antagonist is a C3 polypeptide, a C3-like polypeptide, or a biologically active fragment thereof. In other embodiments, the Rho antagonist is a conjugate comprising a C3 polypeptide, a C3-like polypeptide, or a biologically active fragment thereof. For example, the conjugate is a fusion protein comprising a C3 polypeptide, a C3-like polypeptide, or a biologically active fragment thereof. In yet other embodiments, the conjugate comprises the amino acid sequence of SEQ ID NO:1.

In some embodiments, the bone cell is a bone remodeling cell, e.g., an osteoblast or an osteoblast precursor cell. In other embodiments, the bone remodeling cell is an osteoclast or an osteoclast precursor cell.

In another aspect, the invention features a method of promoting or stimulating bone formation in a subject, and the method comprises administering to the subject a therapeutically effective amount of a Rho antagonist, thereby promoting or stimulating bone formation in the subject.

In some embodiments, the Rho antagonist is a C3 polypeptide, a C3-like polypeptide, or a biologically active fragment thereof. In other embodiments, the Rho antagonist is a conjugate comprising a C3 polypeptide, a C3-like polypeptide, or a biologically active fragment thereof. For example, the conjugate is a fusion protein comprising a C3 polypeptide, a C3-like polypeptide, or a biologically active fragment thereof. In yet other embodiments, the conjugate comprises the amino acid sequence of SEQ ID NO: 1.

In some embodiments, the Rho antagonist is administered at a dosage from about 0.01 pg/kg body weight to about 1 mg/kg body weight, e.g., from about 0.01 pg/kg body weight to about 0.01 mg/kg body weight, from about 0.01 ng/kg body weight to about 0.01 mg/kg body weight, or from about 0.01 μg/kg body weight to about 0.01 mg/kg body weight. In other embodiments, the Rho antagonist is administered at a concentration from about 1 nM to 1 M, e.g., from about 1 μM to 100 mM, from about 0.1 mM to about 100 mM, from about 0.1 mM to about 10 mM, or from about 0.1 mM to about 2 mM. In some embodiments, the Rho antagonist is administered at a concentration of about 0.1 mM, about 0.5 mM, about 1.0 mM, about 10 mM, about 50 mM, about 75 mM, about 100 mM, or about 500 mM.

In other embodiments, the method comprises administering the Rho antagonist in combination with one or more additional agents, e.g., epidermal growth factor (EGF), fibroblast growth factor (FGF), platelet-derived growth factor (PDGF), transforming growth factors (TGFs), parathyroid hormone (PTH), leukemia inhibitory factor (LIF), insulin-like growth factors (IGFs), a bone morphogenetic protein, osteogenin, NaF, estrogen, bisphosphonates and/or calcitonin.

In some embodiments, the subject has a bone injury. In certain embodiments, the bone injury is a bone fracture. In other embodiments, the bone injury is a spinal injury. In yet other embodiments, the bone injury is bone loss incident to a metastasizing tumor.

In another aspect, the invention features a method of treating a bone injury in a subject, comprising administering to the subject a therapeutically effect amount of a Rho antagonist, thereby treating the bone injury.

In some embodiments, the Rho antagonist is a C3 polypeptide, a C3-like polypeptide, or a biologically active fragment thereof. In other embodiments, the Rho antagonist is a conjugate comprising a C3 polypeptide, a C3-like polypeptide, or a biologically active fragment thereof. For example, the conjugate is a fusion protein comprising a C3 polypeptide, a C3-like polypeptide, or a biologically active fragment thereof. In yet other embodiments, the conjugate comprises the amino acid sequence of SEQ ID NO: 1.

In some embodiments, the Rho antagonist is administered at a dosage from about 0.01 pg/kg body weight to about 1 mg/kg body weight, e.g., from about 0.01 pg/kg body weight to about 0.01 mg/kg body weight, from about 0.01 ng/kg body weight to about 0.01 mg/kg body weight, or from about 0.01 μg/kg body weight to about 0.01 mg/kg body weight. In other embodiments, the Rho antagonist is administered at a concentration from about 1 nM to 1 M, e.g., from about 1 μM to 100 mM, from about 0.1 mM to about 100 mM, from about 0.1 mM to about 10 mM, or from about 0.1 mM to about 2 mM. In some embodiments, the Rho antagonist is administered at a concentration of about 0.1 mM, about 0.5 mM, about 1.0 mM, about 10 mM, about 50 mM, about 75 mM, about 100 mM, or about 500 mM.

In some embodiments, the bone injury is a bone fracture. In other embodiments, the bone injury is a spinal injury. In yet other embodiments, the bone injury is bone loss incident to a metastasizing tumor.

In some embodiments, the Rho antagonist is administered locally to the site of the bone injury. In some embodiments, the Rho antagonist is administered locally using a tissue sealant, e.g., a fibrin sealant.

In some embodiments, the Rho antagonist is administered at a dosage of from about 0.001 μg/cm³ tissue to about 50 μg/cm³ tissue. In other embodiments, the Rho antagonist is administered at a dosage of from about 0.0001 μg/cm³ tissue to about 100 μg/cm³ tissue. In yet other embodiments, the Rho antagonist is administered at a dosage of from about 1 μg/cm³ tissue to about 10 μg/cm³ tissue. In yet other embodiments, the Rho antagonist is administered at a dosage of about 50 μg/cm³ tissue.

In certain embodiments, the Rho antagonist is administered at a dosage of about 1 μg in a fibrin sealant. In some embodiments, the fibrin sealant is TISSEEL®. In yet other embodiments, the Rho antagonist is administered directly to bone tissue using a drug eluting medical device.

In other embodiments, the method comprises administering the Rho antagonist in combination with one or more additional agents, e.g., epidermal growth factor (EGF), fibroblast growth factor (FGF), platelet-derived growth factor (PDGF), transforming growth factors (TGFs), parathyroid hormone (PTH), leukemia inhibitory factor (LIF), insulin-like growth factors (IGFs), a bone morphogenetic protein, osteogenin, NaF, estrogen, bisphosphonates and/or calcitonin.

In yet another aspect, the invention features a method of stimulating an osteoblast precursor cell to differentiate into an osteoblast, comprising contacting the osteoblast precursor cell with a Rho antagonist, thereby stimulating the differentiation of the precursor cell.

In some embodiments, the Rho antagonist is a C3 polypeptide, a C3-like polypeptide, or a biologically active fragment thereof. In other embodiments, the Rho antagonist is a conjugate comprising a C3 polypeptide, a C3-like polypeptide, or a biologically active fragment thereof. For example, the conjugate is a fusion protein comprising a C3 polypeptide, a C3-like polypeptide, or a biologically active fragment thereof. In yet other embodiments, the conjugate comprises the amino acid sequence of SEQ ID NO:1.

In another aspect, the invention features a method of inhibiting bone resorption in a subject, comprising administering to the subject a Rho antagonist, thereby inhibiting bone resorption.

In some embodiments, the Rho antagonist is a C3 polypeptide, a C3-like polypeptide, or a biologically active fragment thereof. In other embodiments, the Rho antagonist is a conjugate comprising a C3 polypeptide, a C3-like polypeptide, or a biologically active fragment thereof. For example, the conjugate is a fusion protein comprising a C3 polypeptide, a C3-like polypeptide, or a biologically active fragment thereof. In yet other embodiments, the conjugate comprises the amino acid sequence of SEQ ID NO: 1.

In some embodiments, the Rho antagonist is administered at a dosage from about 0.01 pg/kg body weight to about 1 mg/kg body weight, e.g., from about 0.01 pg/kg body weight to about 0.01 mg/kg body weight, from about 0.01 ng/kg body weight to about 0.01 mg/kg body weight, or from about 0.01 μg/kg body weight to about 0.01 mg/kg body weight. In other embodiments, the Rho antagonist is administered at a concentration from about 1 nM to 1 M, e.g., from about 1 μM to 100 mM, from about 0.1 mM to about 100 mM, from about 0.1 mM to about 10 mM, or from about 0.1 mM to about 2 mM. In some embodiments, the Rho antagonist is administered at a concentration of about 0.1 mM, about 0.5 mM, about 1.0 mM, about 10 mM, about 50 mM, about 75 mM, about 100 mM, or about 500 mM.

In other embodiments, the method comprises administering the Rho antagonist in combination with one or more additional agents, e.g., epidermal growth factor (EGF), fibroblast growth factor (FGF), platelet-derived growth factor (PDGF), transforming growth factors (TGFs), parathyroid hormone (PTH), leukemia inhibitory factor (LIF), insulin-like growth factors (IGFs), a bone morphogenetic protein, osteogenin, NaF, estrogen, bisphosphonates and/or calcitonin.

In yet another aspect, the invention features a method of treating a bone injury in a subject, comprising obtaining osteoblast precursor cells; contacting the cells with a Rho antagonist, thereby stimulating the cells to differentiate into osteoblasts; and implanting the osteoblasts into the subject.

In some embodiments, the Rho antagonist is a C3 polypeptide, a C3-like polypeptide, or a biologically active fragment thereof. In other embodiments, the Rho antagonist is a conjugate comprising a C3 polypeptide, a C3-like polypeptide, or a biologically active fragment thereof. For example, the conjugate is a fusion protein comprising a C3 polypeptide, a C3-like polypeptide, or a biologically active fragment thereof. In yet other embodiments, the conjugate comprises the amino acid sequence of SEQ ID NO:1.

In some embodiments, the Rho antagonist is administered at a dosage from about 0.01 pg/kg body weight to about 1 mg/kg body weight, e.g., from about 0.01 pg/kg body weight to about 0.01 mg/kg body weight, from about 0.01 ng/kg body weight to about 0.01 mg/kg body weight, or from about 0.01 μg/kg body weight to about 0.01 mg/kg body weight. In other embodiments, the Rho antagonist is administered at a concentration from about 1 nM to 1 M, e.g., from about 1 μM to 100 mM, from about 0.1 mM to about 100 mM, from about 0.1 mM to about 10 mM, or from about 0.1 mM to about 2 mM. In some embodiments, the Rho antagonist is administered at a concentration of about 0.1 mM, about 0.5 mM, about 1.0 mM, about 10 mM, about 50 mM, about 75 mM, about 100 mM, or about 500 mM.

In another aspect, the invention features a method of promoting or stimulating chondrocyte differentiation in a subject, comprising administering to the subject a therapeutically effect amount of a Rho antagonist, thereby promoting or stimulating chondrocyte differentiation.

In some embodiments, the Rho antagonist is a C3 polypeptide, a C3-like polypeptide, or a biologically active fragment thereof. In other embodiments, the Rho antagonist is a conjugate comprising a C3 polypeptide, a C3-like polypeptide, or a biologically active fragment thereof. For example, the conjugate is a fusion protein comprising a C3 polypeptide, a C3-like polypeptide, or a biologically active fragment thereof. In yet other embodiments, the conjugate comprises the amino acid sequence of SEQ ID NO:1.

In some embodiments, the Rho antagonist is administered at a dosage from about 0.01 pg/kg body weight to about 1 mg/kg body weight, e.g., from about 0.01 pg/kg body weight to about 0.01 mg/kg body weight, from about 0.01 ng/kg body weight to about 0.01 mg/kg body weight, or from about 0.01 μg/kg body weight to about 0.01 mg/kg body weight. In other embodiments, the Rho antagonist is administered at a concentration from about 1 nM to 1 M, e.g., from about 1 μM to 100 mM, from about 0.1 mM to about 100 mM, from about 0.1 mM to about 10 mM, or from about 0.1 mM to about 2 mM. In some embodiments, the Rho antagonist is administered at a concentration of about 0.1 mM, about 0.5 mM, about 1.0 mM, about 10 mM, about 50 mM, about 75 mM, about 100 mM, or about 500 mM.

In some embodiments, the subject is in need of cartilage regeneration. For example, the subject has a cartilage-related disorder, such as a chondrodysplasia, or has cartilage damaged by trauma or disease, e.g., rheumatoid arthritis or osteoarthritis.

In some embodiments, the Rho antagonist is administered at a dosage of from about 0.001 μg/cm3 tissue to about 50 μg/cm3 tissue. In other embodiments, the Rho antagonist is administered at a dosage of from about 0.0001 μg/cm3 tissue to about 100 μg/cm3 tissue. In yet other embodiments, the Rho antagonist is administered at a dosage of from about 1 μg/cm3 tissue to about 10 μg/cm3 tissue. In yet other embodiments, the Rho antagonist is administered at a dosage of about 50 μg/cm3 tissue.

In certain embodiments, the Rho antagonist is administered at a dosage of about 1 μg in a fibrin sealant. In some embodiments, the fibrin sealant is TISSEEL®. In yet other embodiments, the Rho antagonist is administered directly to bone tissue using a drug eluting medical device.

In another aspect, the invention features a kit comprising a Rho antagonist described herein and instructions for using the Rho antagonist to treat a bone disorder, e.g., a bone disorder described herein. In some embodiments, the kit includes additional therapeutic agents for treating bone disorders.

In another aspect, the invention features the use of a Rho antagonist in the manufacture of a medicament to treat a bone disorder described herein. In some embodiments, the medicament includes additional therapeutic agents for the treatment of bone disorders.

### BRIEF DESCRIPTION OF THE DRAWINGS

The foregoing and other objects of the present invention, the various features thereof, as well as the invention itself, may be more fully understood from the following description, when read together with the accompanying drawings, in which:

FIG. 1 is a graphic representation of thymidine incorporation by primary fetal rat calvarial osteoblast cultures after treatment with control or various concentrations of Cethrin®.

FIG. 2A are representations of photomicrographs of primary fetal rat calvarial osteoblast cultures after treatment with control or various concentrations of Cethrin®.

FIG. 2B are representations of photomicrographs of primary fetal rat calvarial osteoblast cultures after treatment with control or various concentrations of citrate buffer.

FIG. 3 is graphic representation of thymidine incorporation by MCT3T-E1 osteoblast-like cultures after treatment with control or various concentrations of Cethrin®.

FIG. 4 is a graphic representation of thymidine incorporation by actively-growing primary fetal rat calvarial osteoblast cultures after treatment with control or various concentrations of Cethrin®.

FIG. 5A are representations of photomicrographs of actively-growing primary fetal rat calvarial osteoblast cultures after treatment with control or various concentrations of Cethrin®.

FIG. 5B are representations of photomicrographs of actively-growing primary fetal rat calvarial osteoblast cultures after treatment with control or various concentrations of citrate buffer.

FIG. 6 is a graphic representation of thymidine incorporation by primary fetal rat calvarial osteoblast cultures after treatment with control or various concentrations of Cethrin® and various amounts of FCS.

FIG. 7 is a graphic representation of the number of osteoclasts formed from mouse bone marrow cultures following treatment with control or various concentrations of Cethrin®.

FIG. 8 are representations of photomicrographs of mouse bone marrow cultures following treatment with control or various concentrations of Cethrin®.

### DETAILED DESCRIPTION OF THE INVENTION

All publications, patent applications, patents, and other references mentioned herein are incorporated by reference in their entirety. In addition, the materials, methods, and examples are illustrative only and not intended to be limiting. Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below.

The present invention relates to the treatment of bone disorders with Rho antagonists.

### DEFINITIONS

For convenience, certain terms employed in the specification, examples, and appended claims are collected here. Unless defined otherwise, all technical and scientific terms used herein have the same meaning as commonly understood by one of ordinary skill in the art to which this invention belongs. The initial definition provided for a group or term herein applies to that group or term throughout the present specification individually or as part of another group, unless otherwise indicated.

The articles “a” and “an” are used herein to refer to one or to more than one (i.e., to at least one) of the grammatical object of the article. By way of example, “an element” means one element or more than one element.

The term “or” is used herein to mean, and is used interchangeably with, the term “and/or,” unless context clearly indicates otherwise.

The term “about” is used herein to mean a value − or +20% of a given numerical value. Thus, “about 60%” means a value of between 60−(20% of 60) and 60+(20% of 60) (i.e., between 48 and 70).

The term “isolated” is used herein to mean purified to a state beyond that in which it exists in nature. For example an isolated compound can be substantially free of cellular material or other contaminating materials from the cell from which the compound is derived, or substantially free from chemical precursors or other chemicals when chemically synthesized. In some embodiments, the preparation of a compound having less than about 50% (by dry weight) of contaminating materials from the cell, or of chemical precursors is considered to be substantially pure. In other embodiments, the preparation of a compound having less than about 40%, about 30%, about 20%, about 10%, about 5%, about 1% (by dry weight) of contaminating materials from the cell, or of chemical precursors is considered to be substantially pure.

The term “Rho antagonist”, as used herein, means an agent that directly or indirectly inhibits the activity of a Rho GTPase, and/or reduces the level of expression of a Rho GTPase polypeptide.

The term “C3 polypeptide”, as used herein, refers to an ADP-ribosyl transferase C3 polypeptide. Exoenzyme C3 transferase is an ADP ribosyl transferase that selectively ribosylates Rho, rendering it inactive. Such C3 polypeptides are known in the art and include, e.g., C3 polypeptides from Clostridium botulinum, Clostridium limosum, Bacillus cereus or Staphylococcus aureus. The term “C3 polypeptide” also includes recombinant ADP-ribosyl transferase C3 polypeptides.

The term “C3-like polypeptide”, as used herein, refers to any polypeptide having a biological activity similar to (e.g., substantially similar to or the same as) an ADP-ribosyl transferase C3 polypeptide. “C3-like polypeptides” include analogs and derivatives of C3 polypeptides that retain the ability to ADP-ribosylate Rho. In general, the term refers to polypeptides having native C3 polypeptide sequences and structures and having one or more amino acid additions, substitutions and/or deletions, relative to the native polypeptide, so long as the C3-like polypeptides retain C3 polypeptide activity, e.g., ADP-ribosylation activity. C3-like polypeptides can be made from mutant C3 polynucleotide sequences. Examples of C3-like polypeptides include, e.g., those described in U.S. application Ser. Nos. 11/643,940 and 11/808,733.

The terms “peptide”, “polypeptide” and “protein” are used interchangeably herein.

The term “conjugate”, as used herein, means a Rho antagonist that includes a first polypeptide (or a biologically active fragment thereof) that is chemically linked to a heterologous polypeptide, or a first polypeptide (or a biologically active fragment thereof) that is fused to a heterologous polypeptide (e.g., a fusion protein).

“Heterologous polypeptide”, as used herein, is a polypeptide that is different from a first polypeptide.

The term “bone cell” refers to a cell that is involved in the formation, maintenance and homeostasis, remodeling, and structural integrity of bone. Examples of bone cells include but are not limited to osteoblasts, osteoclasts, osteocytes (i.e., quiescent osteoblasts), and osteoprogenitor cells.

The term “bone remodeling” includes the formation, maintenance and homeostasis, remodeling, and structural integrity of bone osteoid, a matrix of collagen and calcium salts that involves first, bone resorption (osteoclastic activity) and second, reactive bone formation (osteoblastic activity). The term “bone remodeling” may refer to normal maintenance of bone homeostasis and to the production of bone in responses to a variety of stress, trauma, injury, and bone disease (e.g., fracture, bone graft, periodontal erosion).

The term “bone remodeling cell” refers to a cell involved in the formation, maintenance and homeostasis, remodeling, and structural integrity of bone osteoid, a matrix of collagen and calcium salts that involves first, bone resorption, and second, reactive bone formation. Examples of bone remodeling cells include, but are not limited to osteoblasts, osteoclasts, osteocytes, and osteoprogenitor cells.

The term “bone disorder” or “skeletal disorder” refers to any condition affecting the development and/or the structure of the skeletal system. Bone disorders can include any disorder that results in an imbalance in the ratio of bone formation to bone resorption such that, if unmodified, a subject will exhibit less bone than desirable, or a subject's bones will be less intact and coherent than desired. Bone disorders may refer to disorders, diseases, conditions or traumas of bone, such as fractures, osteoporosis, osteoarthritis, or cancers. For example, a bone disorder may result from fracture, from surgical intervention or from dental or periodontal disease. These diseases have a variety of causes and symptoms known in the art. Bone disorders can affect people of all ages, from newborns to older adults. However, certain age groups are more likely to have certain types of disorders.

As used herein, the term “precursor cell” refers to a cell that is committed to a differentiation pathway, but that generally does not express markers or function as a mature, fully differentiated cell.

The term “pharmaceutically effective amount” or “therapeutically effective amount” refers to an amount (e.g., dose) effective in treating a patient, having a disorder or condition described herein. It is also to be understood herein that a “pharmaceutically effective amount” may be interpreted as an amount giving a desired therapeutic effect, either taken in one dose or in any dosage or route, taken alone or in combination with other therapeutic agents. For example, a “pharmaceutically effective amount” may be understood to be an amount of a Rho antagonist, e.g., a C3 or C3-like polypeptide, that may, e.g., inhibit Rho activity, e.g., Rho GTPase activity.

The term “treatment” or “treating”, as used herein, refers to administering a therapy in an amount, manner, and/or mode effective to improve a condition, symptom, or parameter associated with a disorder or condition or to prevent or reduce progression of a disorder or condition, either to a statistically significant degree or to a degree detectable to one skilled in the art. An effective amount, manner, or mode can vary depending on the subject and may be tailored to the subject.

The term “subject”, as used herein, means a subject in need of a treatment. For example, a subject can be a mammal, e.g., a human or non-human primate, a cat, a dog, a horse, a cow, or a deer.

### Rho Antagonists

The methods described herein involve the use, such as the administration of, Rho antagonists. A Rho antagonist can be a small molecule, protein (e.g., antibody), peptide, nucleic acid, aptamer, siRNA, or any agent that binds to a Rho family member to inactivate a Rho signaling pathway, or that reduces the expression of a Rho family member.

The Rho family of small GTPases comprises 23 genes in humans, encoding at least 26 different proteins (see, e.g., Bustelo et al., Bioessays 29:356-370, 2007). The Rho family includes, but is not limited to, rho, rac, and cdc42 as well as their isotypes, such as RhoA, RhoB, and RhoC. Rho family members are well-characterized (see, e.g., Takai et al., Physiol. Rev. 81:153-208, 2001; Bishop et al., Biochem. J. 348: 241-255, 2000; Johnson, Microbiol. Mol. Biol. Rev. 63:54-105, 1999; Wennerberg et al., J. Cell Sci. 117:1301-1312, 2004; Burridge et al., Cell 116:167-179, 2004); and their sequences are known (see, e.g., Chardin et al., Nucleic Acids Res. 16:2717, 1988; Yeramian et al., Nucleic Acids Res. 15:1869, 1987).

In some instances, a Rho antagonist is used to modulate Rho GTPase activity. The amount of a Rho antagonist that modulates Rho GTPase activity can be measured using standard assays known in the art (see, e.g., Rojas et al., Comb. Chem. High Throughput Screen. 6:409-418 (2003)).

Bacterial Protein Antagonists

Rho antagonists that are useful in the methods described herein include, but are not limited to, bacterial proteins known to inhibit Rho. For example, a bacterial ADP ribosyltransferase, C3 transferase, ribosylates Rho to inactivate the protein (see, e.g., Tominaga et al., Meth. Enzymol. 256:290-297, 1995; Boquet et al., Meth. Enzymol. 256:297-306, 1995; Lang et al., Meth. Enzmmol. 256: 320-327, 1995; Stasia et al., Meth. Enzymol. 256: 327-336, 1995). C3 polypeptides include, e.g., C3 polypeptides from Clostridium botulinum, Clostridium limosum, Bacillus cereus and Staphylococcus aureus. Any known C3 polypeptides can be used in the methods described herein. Likewise, other bacterial toxins, such as toxins A and B, with related Rho-inhibitory activity can also be used as Rho antagonists in the methods described herein.

In some instances, Rho antagonists include, e.g., C3 polypeptides or biologically active fragments thereof, C3-like polypeptides or biologically active fragments thereof, and conjugate proteins that include C3 or C3-like polypeptides or biologically active fragments thereof (e.g., C3 polypeptide fusion proteins).

For example, in some nonlimiting instances, the Rho antagonist is a conjugate that includes a C3 or a C3-like polypeptide (or a biologically active fragment thereof), and a heterologous polypeptide. In some examples, the heterologous polypeptide is a transport agent that facilitates cellular uptake of the conjugate. Nonlimiting exemplary conjugates are described in U.S. application Ser. Nos. 11/643,940 and 11/808,733.

Another nonlimiting exemplary conjugate is the fusion protein BA-210 (C3-11), described as SEQ ID NO:10 in U.S. application Ser. Nos. 11/643,940 and 11/808,733. BA 210 is composed of 232 amino acids, with a theoretical molecular weight of 25,858 daltons and a theoretical isoelectric point (pI) of 9.6. BA-210 is the active pharmaceutical ingredient in Cethrin® (BioAxone Therapeutic, Inc., Montreal, Canada), and has been engineered in part through the addition of a proline rich peptidic transport sequence to a C3 exoenzyme sequence (see Winton et al., J. Biol. Chem. 277:32820-32829, 2002). Various analogs and derivates of BA-210 are also useful in the methods described herein.

Antisense and Ribozymes

Other Rho antagonists that are useful in the methods described herein are nucleic acid antagonists, including antisense molecules or catalytic nucleic acid molecules (e.g., ribozymes) that specifically hybridize mRNA encoding a Rho family member. An antisense construct includes the reverse complement of at least part of the cDNA coding sequence or mRNA of a Rho family member, the Rho family member cDNA, or gene sequence or flanking regions thereof, and thus it can hybridize to the mRNA.

The introduced sequence need not be the full-length cDNA or gene or reverse complement thereof, and need not be exactly homologous to the equivalent sequence found in the cell type to be transformed. Antisense molecules can be made using known techniques in the art (see, e.g., Agrawal, Methods in Molecular Biology, Humana Press Inc., 1993, Vol. 20 (“Protocols for Oligonucleotides and Analogs”)).

The antisense molecule may be conjugated to another molecule, e.g., a peptide, hybridization triggered cross-linking agent, transport agent, aptamer, or hybridization-triggered cleavage agent. A targeting moiety can also be included that enhances uptake of the molecule by cells, e.g., bone cells. The targeting moiety can be a specific binding molecule, such as an antibody or fragment thereof that recognizes a molecule present on the surface of the cell, e.g., bone cell.

Alternatively, the Rho antagonist is a catalytic nucleic acid, such as a ribozyme (a synthetic RNA molecule that possesses highly specific endoribonuclease activity). The production and use of ribozymes are disclosed in, e.g., U.S. Pat. No. 4,987,071 and U.S. Pat. No. 5,543,508. Ribozymes can be synthesized and administered to a cell or a subject, or can be encoded on an expression vector, from which the ribozyme is synthesized in the targeted cell (see, e.g., PCT Pub. WO 9523225, and Beigelman et al., Nucl. Acids Res. 23:4434-42, 1995). Examples of oligonucleotides with catalytic activity are described in, e.g., PCT Publication Nos. WO 9506764 and WO 9011364, and Sarver et al., Science 247:1222-1225, 1990. The inclusion of ribozyme sequences within antisense RNAs can be used to confer RNA cleaving activity on the antisense RNA, such that endogenous mRNA molecules that bind to the antisense RNA are cleaved, which, in turn, leads to an enhanced antisense inhibition of endogenous gene expression.

RNA Interference

Double-stranded nucleic acid molecules that can silence a gene encoding a Rho family member can also be used as Rho antagonists. RNA interference (RNAi) is a mechanism of post-transcriptional gene silencing in which double-stranded RNA (dsRNA) corresponding to a gene (or coding region) of interest is introduced into a cell or an organism, resulting in degradation of the corresponding mRNA. The RNAi effect persists for multiple cell divisions before gene expression is regained. RNAi is therefore an effective method for making targeted knockouts or “knockdowns” at the RNA level. RNAi has proven successful in human cells, including human embryonic kidney and HeLa cells (see, e.g., Elbashir et al., Nature 411:494-498, 2001). For example, gene silencing can be induced in mammalian cells by the endogenous expression of RNA hairpins (see Paddison et al., PNAS (USA) 99:1443-1448, 2002). In another instances, transfection of small (21-23 nt) dsRNA specifically inhibits gene expression (reviewed in Caplen, Trends in Biotechnol. 20:49-51, 2002).

Briefly, RNAi is thought to work as follows. miRNA, pre-miRNA, pri-miRNA, or dsRNA corresponding to a portion of a gene to be silenced is introduced into a cell. The dsRNA is digested into 21-23 nucleotide siRNAs, or short interfering RNAs. The siRNA duplexes bind to a nuclease complex to form what is known as the RNA-induced silencing complex, or RISC. The RISC targets the homologous transcript by base pairing interactions between one of the siRNA strands and the endogenous mRNA. It then cleaves the mRNA approximately 12 nucleotides from the 3′ terminus of the siRNA (reviewed in Sharp et al., Genes Dev. 15: 485-490, 2001; and Hammond et al., Nature Rev. Gen. 2: 110-119, 2001).

RNAi technology in gene silencing utilizes standard molecular biology methods. dsRNA corresponding to the sequence from a target gene to be inactivated can be produced by standard methods, e.g., by simultaneous transcription of both strands of a template DNA (corresponding to the target sequence) with T7 RNA polymerase. Kits for production of dsRNA for use in RNAi are available commercially, e.g., from New England Biolabs, Inc. Methods of transfection of dsRNA or plasmids engineered to make dsRNA are routine in the art.

Gene silencing effects similar to those of RNAi have been reported in mammalian cells with transfection of a mRNA-cDNA hybrid construct (Lin et al., Biochem. Biophys. Res. Commun. 281:639-644, 2001), and can be used as another method for gene silencing. Therapeutic applications of RNAi are described, e.g., in Shuey, Drug Discov. Today 7:1040-1046, 2002.

Aptamers

In some instances, the Rho antagonist is an aptamer that targets a Rho family member. Aptamers are nucleic acid molecules having a tertiary structure that permits them to specifically bind to protein ligands (see, e.g., Osborne et al., Curr. Opin. Chem. Biol. 1:5-9, 1997; and Patel, Curr. Opin. Chem. Biol. 1:32-46, 1997). Aptamers can also be conjugated to siRNA or miRNA (see WO 2007/143086).

Rho-targeted aptamers may be created using a type of in vitro natural selection for randomly-generated nucleic acid sequences that bind to the selected target. This method has been termed “SELEX” (for Systematic Evolution of Ligands by Exponential Enrichment). The SELEX method (hereinafter termed SELEX) and related application are described in, e.g., U.S. Pat. No. 5,475,096, U.S. Pat. No. 6,083,696, U.S. Pat. No. 6,441,158 and U.S. Pat. No. 6,458,559. The SELEX process provides a class of products that are referred to as nucleic acid ligands, such ligands having a unique sequence, and that have the property of binding specifically to a desired target compound or molecule. Each SELEX-identified nucleic acid ligand is a specific ligand of a given target compound or molecule. SELEX is based on the insight that nucleic acids have sufficient capacity for forming a variety of two- and three-dimensional structures and sufficient chemical versatility available within their monomers to act as ligands (form specific binding pairs) with virtually any chemical compound, whether monomeric or polymeric.

Briefly, the SELEX method involves selection from a mixture of candidates and step-wise iterations of binding, partitioning, and amplification, using the same general selection theme, to achieve virtually any desired criterion of binding affinity and selectivity. Starting from a mixture of nucleic acids, preferably comprising a segment of randomized sequence, the method includes contacting the mixture with the target under conditions favorable for binding, partitioning unbound nucleic acids from those nucleic acids which have bound to target molecules, dissociating the nucleic acid-target pairs, amplifying the nucleic acids dissociated from the nucleic acid-target pairs to yield a ligand-enriched mixture of nucleic acids, then reiterating the steps of binding, partitioning, dissociating and amplifying through as many cycles as desired. A variety of techniques can be used to partition members in the pool of nucleic acids that have a higher affinity to the target than the bulk of the nucleic acids in the mixture.

While not bound by theory, SELEX is based on the observation that within a nucleic acid mixture containing a large number of possible sequences and structures there is a wide range of binding affinities for a given target. A nucleic acid mixture comprising, for example, a 20-nucleotide randomized segment, can have 420 candidate possibilities. Those that have the higher affinity constants for the target are most likely to bind to the target. After the partitioning, dissociating and amplifying steps, a second nucleic acid mixture is generated, enriched for the higher binding affinity candidates. Additional rounds of selection progressively favor the best ligands until the resulting nucleic acid mixture is predominantly composed of only one or a few sequences. These can then be cloned, sequenced and individually tested for binding affinity as pure ligands.

Cycles of selection, partition and amplification are repeated until a desired goal is achieved. In the most general case, selection, partition and amplification is continued until no significant improvement in binding strength is achieved on repetition of the cycle. The method may be used to sample as many as about 1018 different nucleic acid species. The nucleic acids of the test mixture preferably include a randomized sequence portion as well as conserved sequences necessary for efficient amplification. Nucleic acid sequence variants can be produced in a number of ways including synthesis of randomized nucleic acid sequences and size selection from randomly cleaved cellular nucleic acids. The variable sequence portion may contain fully or partially random sequence; it may also contain subportions of conserved sequence incorporated with randomized sequence. Sequence variation in test nucleic acids can be introduced or increased by mutagenesis before or during the selection, partition and amplification iterations.

The basic SELEX method may be modified to achieve specific objectives. For example, U.S. Pat. No. 5,707,796, describes the use of SELEX in conjunction with gel electrophoresis to select nucleic acid molecules with specific structural characteristics, such as bent DNA. U.S. Pat. No. 5,763,177, describes a SELEX based method for selecting nucleic acid ligands containing photoreactive groups capable of binding and/or photocrosslinking to and/or photoinactivating a target molecule. U.S. Pat. No. 5,580,737, describes a method for identifying highly specific nucleic acid ligands able to discriminate between closely related molecules, termed “counter-SELEX”. U.S. Pat. No. 5,567,588, describes a SELEX-based method which achieves highly efficient partitioning between oligonucleotides having high and low affinity for a target molecule.

The SELEX method encompasses the identification of high-affinity nucleic acid ligands containing modified nucleotides conferring improved characteristics on the ligand, such as improved in vivo stability or delivery. Examples of such modifications include chemical substitutions at the ribose and/or phosphate and/or base positions. Specific SELEX-identified nucleic acid ligands containing modified nucleotides are described in, e.g., U.S. Pat. No. 5,660,985, which describes oligonucleotides containing nucleotide derivatives chemically modified at the 5- and 2′-positions of pyrimidines, as well as specific RNA ligands to thrombin containing 2′-amino modifications. Also included are highly specific nucleic acid ligands containing one or more nucleotides modified with 2′-amino (2′-NH₂), 2′-fluoro (2′-F), and/or 2′-O-methyl (2′-OMe).

Dominant Negative Mutants

In some instances, the Rho antagonist useful in the methods described herein is a dominant negative mutant of a Rho family member. In general, a dominant negative protein is a mutated or otherwise altered derivative of a native protein that is capable of interfering with the function of the native protein. A dominant negative Rho antagonist refers to a mutated Rho family gene and/or encoded mutated Rho family protein that lacks the activity of the wild-type Rho family protein but that inhibits the activity of the wild-type Rho family protein when co-expressed along with the wild-type protein.

Dominant negative forms of Rho family proteins are known in the art and include, without limitation, RhoA (N19) (see, e.g., Khosravi-Far et al., Mol. Cell. Biol. 15:6443-6453, 1995); RhoB (N19) (see, e.g., Prendergast et al., Oncogene 10:2289-2296, 1995); and Rac1 (N17) (see, e.g., Khosravi-Far et al., Mol. Cell. Biol. 15:6443-6453, 1995; and Qiu et al., Nature 374:457-459, 1995). Another Rho antagonist is Rho with a mutated effector domain, A-37, which prevents GTP exchange.

Other Rho antagonists include fragments of Rho family members that incorporate the ectodomain, including the ectodomain per se and other N- and/or C-terminally truncated fragments of Rho family members or the ectodomain, as well as analogs thereof in which amino acids, e.g., from 1 to 10 amino acids, are substituted, particularly conservatively, and derivatives of Rho family members or Rho family members fragments in which the N- and/or C-terminal residues are derivatized by chemical stabilizing groups. Rho family fragments can be produced either by peptide synthesis or by recombinant DNA expression (using standard recombinant procedures) of either a truncated Rho protein or of an intact Rho protein that is subsequently digested enzymatically in either a random or a site-selective manner. Analogs of Rho family members or Rho family members fragments can be generated also by recombinant DNA techniques or by peptide synthesis, and can incorporate one or more, e.g., 1 to 5, L- or D-amino acid substitutions. Derivatives of Rho family members, Rho family members fragments and Rho family members analogs can be generated by chemical reaction of the Rho protein to incorporate the desired derivatizing group, such as N-terminal, C-terminal and intra-residue modifying groups that have the effect of masking or stabilizing the substance or target amino acids within it.

Rho family derivatives, analogs, and fragments can be produced by various methods known in the art. The manipulations, which result in their production, can occur at the gene or protein level. For example, DNA can be modified by any of numerous strategies known in the art (e.g., Maniatis et al., Molecular Cloning, A Laboratory Manual, Cold Spring Harbor Laboratory, Cold Spring Harbor, N.Y., 1982), such as by cleavage at appropriate sites with restriction endonuclease(s), subjected to enzymatic modifications if desired, isolated, and ligated in vitro.

Antibody Antagonists

Rho antagonists that can be used in the methods described herein also include antagonist antibodies directed against Rho family proteins. An antagonist antibody can be, e.g., a polyclonal antibody; a monoclonal antibody or antigen binding fragment thereof; a modified antibody such as a chimeric antibody, reshaped antibody, humanized antibody, or fragment thereof (e.g., Fab′, Fab, F(ab′)₂); or a biosynthetic antibody, e.g., a single chain antibody, single domain antibody (DAB), Fv, single chain Fv (scFv), or the like.

Methods of making and using polyclonal and monoclonal antibodies are described, e.g., in Harlow et al., Using Antibodies: A Laboratory Manual: Portable Protocol I. Cold Spring Harbor Laboratory (Dec. 1, 1998). Methods for making modified antibodies and antibody fragments (e.g., chimeric antibodies, reshaped antibodies, humanized antibodies, or fragments thereof, e.g., Fab′, Fab, F(ab′)₂ fragments); or biosynthetic antibodies (e.g., single chain antibodies, single domain antibodies (DABs), Fv, single chain Fv (scFv), and the like), are known in the art and can be found, e.g., in Zola, Monoclonal Antibodies: Preparation and Use of Monoclonal Antibodies and Engineered Antibody Derivatives, Springer Verlag (Dec. 15, 2000; 1 st edition).

Small Molecule Antagonists

Small molecules that inhibit the activity of Rho family members can be used as Rho antagonists in the methods described herein. Such molecules are known in the art and include, without limitation, CCG-1423 (see, e.g., Evelyn et al., Mol. Cancer. Ther. 6:2249-2260, 2007). Other small molecule antagonists can be identified using routine methods, e.g., as described in Rojas et al., Comb. Chem. High Throughput Screen 6:409-418, 2003.

### Use of Rho Antagonists in Disorders and Injuries

In certain methods described herein, a Rho antagonist is used to treat or prevent a bone disorder or injury. Examples of bone disorders or injuries include, but are not limited to, osteoporosis, secondary osteoporosis, perimenopausal bone loss, osteopenia, osteogenesis imperfecta, osteochondroma, bone tumor, giant cell tumor of bone, osteoid osteoma, osteosarcoma, osteonecrosis, bone spurs, craniosynostosis, enchondroma fibrous dysplasia, infectious arthritis, Klippel-Feil Syndrome, limb length discrepancy, osteitis condensans ilii, osteochondritis dissecans, osteomyelitis, osteopetrosis, renal osteodystrophy, bone cyst, unicameral bone cyst, craniosynostosis, scoliosis, osteolysis, osteomalacia, osteoperiostitis, osteotomy, bone fracture, bone transplantation, degenerative joint disease, osteoarthritis, osteogenesis imperfecta, arthritis, secondary osteoarthritis, Paget disease, Freiberg's disease, Kohler disease, and Rickets.

In some instances, the bone fracture is a complete fracture, for example, a fracture in which bone fragments separate completely. In another instances, the bone fracture is an incomplete fracture, for example, a fracture in which the bone fragments are still partially joined. Additional types of bone fractures include, but are not limited to, linear fracture, for example, a fracture that is parallel to the bone's long axis; transverse fracture, for example, a fracture that is at a right angle to the bone's long axis; oblique fracture, for example, a fracture that is diagonal to a bone's long axis; compression fracture, for example, a fracture that usually occurs in the vertebrae; spiral fracture, for example, a fracture where at least one part of the bone has been twisted; comminuted fracture, for example, a fracture causing many fragments; compacted fracture, for example, a fracture caused when bone fragments are driven into each other; and open fracture, for example, a fracture when the bone reaches the skin.

In other instances, a Rho antagonist described herein can be used in the promotion of bone healing in plastic surgery; stimulation of bone ingrowth into non-cemented prosthetic joints and dental implants; elevation of peak bone mass in pre-menopausal women; treatment of growth deficiencies; treatment of periodontal disease and defects, and other tooth repair processes; increase in bone formation during distraction osteogenesis; and treatment of other skeletal disorders, such as age-related osteoporosis, post-menopausal osteoporosis, glucocorticoid-induced osteoporosis or disuse osteoporosis and arthritis, or any condition that benefits from stimulation of bone formation. The Rho antagonists described herein can also be used in repair of congenital, trauma-induced or surgical resection of bone (for instance, for cancer treatment), and in cosmetic surgery.

Other bone injuries that can be treated with the methods described herein are incident to spinal injuries. For example, current therapies for some spinal injuries include surgically fusing together two or more spinal bones. A Rho antagonist described herein can be administered as an alternative to, or in addition to, such spinal bone fusion therapies.

In yet other instances, the bone loss is periodontal bone loss due to inflammation resulting from, e.g., bacterial infection, ingestion or inhalation of noxious substances, including those from tobacco. For example, a Rho antagonist described herein can be administered to, e.g., around, the root of a tooth, to stimulate the restoration and integrity of bone surrounding the tooth, and around dental implants.

In other instances, a Rho antagonist described herein can be used to induce or promote chondrocyte differentiation, to treat or prevent a chondrocyte-related condition, such as chondrodysplasias, and/or to stimulate cartilage regeneration (see, e.g., Wang et al., J. Biol. Chem. 279:13205-13214, 2004; Woods et al., J. Biol. Chem. 281:13134-13140, 2006). The Rho antagonist can also be used for stimulating chondrocytes, with the potential of cartilage repair, e.g., to treat osteoarthritis and other arthritises and to facilitate autologous bone and cartilage transplants in subjects. Cartilage may develop abnormally or may be damaged by disease, such as rheumatoid arthritis or osteoarthritis, or by trauma, each of which can lead to physical deformity and debilitation. Whether cartilage is damaged from trauma or congenital anomalies, its successful clinical regeneration is often poor at best, as reviewed by Howell et al., Osteoarthritis: Diagnosis and Management, 2nd ed. (Philadelphia, W.B. Saunders, 1990); and Kelley et al., Textbook of Rheumatology, 3rd ed. (Philadelphia, W.B. Saunders, 1989). The Rho antagonists described herein can be used to regenerate damaged cartilage.

### Treatment of Bone Cells and Chondrocytes

In some methods described herein, Rho antagonists are used to stimulate osteoblast differentiation or to stimulate an osteoblast precursor cell to differentiate into an osteoblast. One of ordinary skill in the art can identify osteoblasts and osteoblast precursor cells using known methods and identifying markers.

For example, osteoblasts exhibit known protein markers, such as alkaline phosphatase (ALP) (see, e.g., Ongphiphadhanakul et al., Endocrinol. 133:2502-2507, 1993; Chipoy et al., J. Bone Miner. Res. 19:1850-1861, 2004); alpha 1(I) procollagen (see, e.g., Zhou et al., J. Bone Miner. Res. 9:1489-99, 1994); Bone Gla Protein (BGP) (see, e.g., Zhou et al., J. Bone Miner. Res. 9:1489-99, 1994); Bone Sialoprotein (BSP) (see, e.g., Chen et al., Calcif. Tissue Int. 60:283-90, 1997; Chipoy et al., J. Bone Miner. Res. 19:1850-1861, 2004); Cbfa1/Osf2 (see, e.g., Garcia et al., Bone 31:205-211, 2002); Collagen Type I (see, e.g., Ongphiphadhanakul et al., Endocrinol. 133:2502-2507, 1993; Dacic et al., J. Bone Miner. Res. 16:1228-36, 2001); E11 (see, e.g., Wetterwald et al., Bone 18:125-132, 1996); Osteocalcin (see, e.g., Ongphiphadhanakul et al., Endocrinol. 133:2502-2507, 1993; Chipoy et al., J. Bone Miner. Res. 19:1850-1861, 2004); Osteopontin (see, e.g., Zhou et al., J. Bone Miner. Res. 9:1489-99, 1994; Ongphiphadhanakul et al., Endocrinol. 133:2502-2507, 1993); Phex (see, e.g., Ecarot et al., Endocrinol. 140:1192-1199, 1999); and RP59 (see, e.g., Kruger et al., Dev. Dyn. 223:414-418, 2002).

Osteoclasts can be identified using known markers, e.g., acid ATPase (see, e.g., Andersson et al., Connect. Tissue Res. 20:151-158, 1989); Calcitonin (CT) receptor (CTR) (see, e.g., Lee et al., Endocrinol. 136:4572-4581, 1995; Rouleau et al., J. Bone Miner. Res. 1:543-553, 1986); Carboxyterminal Telopeptide of Type 1 Collagen (1CTP) (see, e.g., Gough et al., Diabet. Med. 14:527-531, 1997); Cathepsin K (see, e.g., Dodds et al., Cell. Biochem. Funct. 21:231-234, 2003; Dodds et al., J. Bone Miner. Res. 16:478-486, 2001); creatine kinase BB (CKBB) (see, e.g., Whyte et al., J. Bone Miner. Res. 11:1438-1443, 1996; Bollerslev et al., Clin. Orthop. Relat. Res. 377:241-247, 2000); acidotrophic amine 3-(2,4-dinitroanillino)-3′-amino-N-methyldipropylamine (DAMP) (see, e.g., Inoue et al., Cell Tissue Res. 298:527-537, 1999); EDI (see, e.g., Wildemann et al., Biomaterials 26:4035-4040, 2005; Miao et al., BMC Musculoskelet. Disord. 3:16, 2002); Kat1-antigen (Kat1-Ag) (see, e.g., Kukita et al., Histochem. Cell Biol. 115:215-222, 2001); procollagen carboxyterminal propeptide (P1CP) (see, e.g., Gough et al., Diabet. Med. 14:527-531, 1997); RANK (see, e.g., Atkins et al., J. Bone Miner. Res. 21:1339-1349, 2006); Tartrate-resistant acid ATPase (see, e.g., Andersson et al., J. Histochem. Cytochem. 37:115-117, 1989); tartrate-resistant acid phosphatise (TRAP) (see, e.g., Ballanti et al., Osteoporos. Int. 7:39-43, 1997; Minkin et al., Calcif. Tissue Int. 34:285-290, 1982); Vacuolar-Type Proton Pump (E11) (see, e.g., Kurihara et al., Endocrinol. 127:3215-3221, 1990); and Vitronectin Receptor (VR, VNR) (see, e.g., Sakiyama et al., J. Bone Miner. Metab. 19:220-227, 2001; Kurihara et al., Endocrinol. 127:3215-3221, 1990).

Osteoblasts or their precursors are identifiable by markers including osteoblast-specific factor-2 (OSF-2), osteoprotegerin (OPG; RANKL), osteopontin (OP), osteocalcin (OC), collagen 1, tartrate-resistant alkaline phosphatase, bone-specific alkaline phosphatise (BAP), and stromal stem cell marker-1 (STR0-1).

In other methods described herein, Rho antagonists are used to stimulate chondrocyte differentiation. One of ordinary skill in the art can identify chondrocytes using known methods and identifying markers. Chondrocyte markers include, without limitation, 11-fibrau (see, e.g., van Osch et al., Biochem. Biophys. Res. Commun. 280:806-812, 2001); aggrecan (see, e.g., Sive et al., Mol. Pathol. 55:91-97, 2002); annexin VI (see, e.g., Pfander et al., Am. J. Pathol. 159:1777-1783, 2001); beta1 integrin (CD29) (see, e.g., Salter et al., J. Histochem. Cytochem. 43:447-457, 1995); COMP (cartilage oligomeric matrix protein) (see, e.g., Zaucke et al., Biochem. J. 358:17-24, 2001); cathepsin B (see, e.g., Baici et al., Ann. Rheum. Dis. 47:684-691, 1988); CEP-68 (see, e.g., Steck et al., Biochem. J. 353:169-174, 2001); collagen II (see, e.g., Sive et al., Mol. Pathol. 55:91-97, 2002); collagen IX (see, e.g., Zaucke et al., Biochem. J. 358:17-24, 2001); collagen X (see, e.g., Wu et al., Exp. Cell. Res. 256:383-391, 2000); MMP13 (see, e.g., D'Angelo et al., J. Cell. Biochem. 77:678-693, 2000); sox9 (see, e.g., Sive et al., Mol. Pathol. 55:91-97, 2002); and syndecan-3 (see, e.g., Pfander et al., Am. J. Pathol. 159:1777-1783, 2001).

### Pharmaceutical Compositions and Administration

The Rho antagonists described herein can be incorporated into pharmaceutical compositions to be used in the methods described herein. Such compositions typically include a Rho antagonist and a pharmaceutically acceptable carrier.

As used herein, a “pharmaceutically acceptable carrier” means a carrier that can be administered to a subject together with a Rho antagonist described herein, which does not destroy the pharmacological activity thereof. Pharmaceutically acceptable carriers include, e.g., solvents, dispersion media, coatings, antibacterial and antifungal agents, isotonic and absorption delaying agents, and the like, compatible with pharmaceutical administration. Supplementary active compounds can also be incorporated into the compositions.

Non-limiting examples of pharmaceutically acceptable carriers that can be used include poly(ethylene-co-vinyl acetate), PVA, partially hydrolyzed poly(ethylene-co-vinyl acetate), poly(ethylene-co-vinyl acetate-co-vinyl alcohol), a cross-linked poly(ethylene-co-vinyl acetate), a cross-linked partially hydrolyzed poly(ethylene-co-vinyl acetate), a cross-linked poly(ethylene-co-vinyl acetate-co-vinyl alcohol), poly-D,L-lactic acid, poly-L-lactic acid, polyglycolic acid, PGA, copolymers of lactic acid and glycolic acid (PLGA), polycaprolactone, polyvalerolactone, poly (anhydrides), copolymers of polycaprolactone with polyethylene glycol, copolymers of polylactic acid with polyethylene glycol, polyethylene glycol; and combinations and blends thereof.

Other carriers include, e.g., an aqueous gelatin, an aqueous protein, a polymeric carrier, a cross-linking agent, or a combination thereof. In another instances, the carrier is a matrix. In yet another instances, the carrier includes water, a pharmaceutically acceptable buffer salt, a pharmaceutically acceptable buffer solution, a pharmaceutically acceptable antioxidant, ascorbic acid, one or more low molecular weight pharmaceutically acceptable polypeptides, a peptide comprising about 2 to about 10 amino acid residues, one or more pharmaceutically acceptable proteins, one or more pharmaceutically acceptable amino acids, an essential-to-human amino acid, one or more pharmaceutically acceptable carbohydrates, one or more pharmaceutically acceptable carbohydrate-derived materials, a non-reducing sugar, glucose, sucrose, sorbitol, trehalose, mannitol, maltodextrin, dextrins, cyclodextrin, a pharmaceutically acceptable chelating agent, EDTA, DTPA, a chelating agent for a divalent metal ion, a chelating agent for a trivalent metal ion, glutathione, pharmaceutically acceptable nonspecific serum albumin, and/or combinations thereof.

A pharmaceutical composition containing a Rho antagonist can be formulated to be compatible with its intended route of administration as known by those of ordinary skill in the art. Nonlimiting examples of routes of administration include parenteral, e.g., intravenous, intradermal, subcutaneous, oral (e.g., inhalation), transdermal (topical), transmucosal, vaginal and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringability exists. It should be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. It may be desirable to include isotonic agents, for example, sugars, polyalcohols such as mannitol, sorbitol, or sodium chloride in the composition. Prolonged absorption of the injectable compositions can be accomplished by including in the composition an agent that delays absorption, for example, aluminum monostearate and gelatin (see, e.g., Remington: The Science and Practice of Pharmacy, 21^{st} edition, Lippincott Williams & Wilkins, Gennaro, ed. (2006)).

Sterile injectable solutions can be prepared by incorporating a Rho antagonist in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, the methods of preparation include, without limitation, vacuum drying and freeze-drying which yields a powder of the active ingredient plus any additional desired ingredient from a previously sterile-filtered solution thereof.

Oral compositions generally include an inert diluent or an edible carrier. For the purpose of oral therapeutic administration, a Rho antagonist can be incorporated with excipients and used in the form of tablets, pills, troches, or capsules, e.g., gelatin capsules. Oral compositions can also be prepared using a fluid carrier for use as a mouthwash. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, a Rho antagonist can be delivered in the form of an aerosol spray from pressured container or dispenser that contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, but are not limited to, for example, for transmucosal administration, detergents, bile salts, and fusidic acid derivatives. Transmucosal administration can be accomplished through the use of nasal sprays or suppositories. For transdermal administration, the active compounds are formulated into, e.g., ointments, salves, gels, or creams as generally known in the art.

The pharmaceutical compositions containing a Rho inhibitor can also be prepared in the form of suppositories (e.g., with conventional suppository bases such as cocoa butter and other glycerides) or retention enemas for rectal delivery.

Alternatively or additionally, a Rho antagonist can be applied locally into a bone or the surrounding tissue, e.g., directly into a disease site, e.g., directly on or into the margins remaining after resection of a bone tumor. For example, a Rho antagonist can be applied using drug eluting or delivery devices known in the art.

Some pharmaceutical compositions can be prepared with a carrier that protects the Rho antagonist against rapid elimination from the body, such as a controlled release formulation, including implants and microencapsulated delivery systems (as described, e.g., in Tan et al., Pharm. Res. 24:2297-2308, 2007). Biodegradable, biocompatible polymers can be used, such as ethylene vinyl acetate, polyanhydrides, polyglycolic acid, collagen, polyorthoesters, and polylactic acid. Methods for preparation of such formulations are apparent to those skilled in the art. The materials can also be obtained commercially (e.g., from Alza Corp., Mountain View, Calif.). Liposomal suspensions (including liposomes targeted to particular cells with monoclonal antibodies to cell surface antigens) can also be used as pharmaceutically acceptable carriers. These can be prepared according to methods known to those skilled in the art, e.g., as described in U.S. Pat. No. 4,522,811.

It may be advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier.

Toxicity and therapeutic efficacy of such compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. While compounds that exhibit toxic side effects can be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds lies generally within a range of circulating concentrations that include the ED50 with little or no toxicity. The dosage can vary within this range depending upon the dosage form employed and the route of administration utilized. For any compound used in the method of the invention, the therapeutically effective dose can be estimated initially from cell culture assays. A dose can be formulated in animal models to achieve a circulating plasma concentration range that includes the IC50 (i.e., the concentration of the test compound which achieves a half-maximal inhibition of symptoms) as determined in cell culture. Such information can be used to more accurately determine useful doses in humans. Levels in plasma can be measured, for example, by high performance liquid chromatography. Information for preparing and testing such compositions are known in the art (see, e.g., Remington: The Science and Practice of Pharmacy, 21^{st} edition, Lippincott Williams & Wilkins, Gennaro, ed. (2006)).

In some instances, a therapeutically effective amount of about 0.1 μg Rho antagonist per cm³ of tissue to about 10,000 μg Rho antagonist per cm³ of tissue is administered, e.g., about 0.1 μg Rho antagonist per cm³ of tissue to about 1,000 μg Rho antagonist per cm³ of tissue, about 0.1 μg Rho antagonist per cm³ of tissue to about 500 μg Rho antagonist per cm³ of tissue, about 0.5 μg Rho antagonist per cm³ of tissue to about 500 μg Rho antagonist per cm³ of tissue, about 1 μg Rho antagonist per cm³ of tissue to about 500 μg Rho antagonist per cm³ of tissue, about 10 μg Rho antagonist per cm³ of tissue to about 100 μg Rho antagonist per cm³ of tissue, about 20 μg Rho antagonist per cm³ of tissue to about 80 μg Rho antagonist per cm³ of tissue, or about 50 μg Rho antagonist per cm³ of tissue.

In other instances, about 0.001 μg Rho antagonist per cm³ of tissue to about 1,000 μg Rho antagonist per cm³ of tissue is administered, e.g., about 0.01 μg Rho antagonist per cm³ of tissue to about 500 μg Rho antagonist per cm³ of tissue, about 0.05 μg Rho antagonist per cm³ of tissue to about 500 μg Rho antagonist per cm³ of tissue, about 0.1 μg Rho antagonist per cm³ of tissue to about 500 μg Rho antagonist per cm³ of tissue, about 0.2 μg Rho antagonist per cm³ of tissue to about 250 μg Rho antagonist per cm³ of tissue, about 0.5 μg Rho antagonist per cm³ of tissue to about 200 μg Rho antagonist per cm³ of tissue, or about 1 μg Rho antagonist per cm³ of tissue to about 100 μg Rho antagonist per cm³ of tissue.

In other instances, about 0.0005 μM Rho antagonist to about 50 μM Rho antagonist is administered, e.g., about 0.005 μM Rho antagonist to about 50 μM Rho antagonist, about 0.01 μM Rho antagonist to about 50 μM Rho antagonist, about 0.02 μM Rho antagonist to about 50 μM Rho antagonist, about 0.03 μM Rho antagonist to about 50 μM Rho antagonist, about 0.04 μM Rho antagonist to about 50 μM Rho antagonist, about 0.05 μM Rho antagonist to about 50 μM Rho antagonist, about 0.1 μM Rho antagonist to about 50 μM Rho antagonist, about 0.1 μM Rho antagonist to about 25 μM Rho antagonist, about 0.2 μM Rho antagonist to about 20 μM Rho antagonist, about 0.5 μM Rho antagonist to about 20 μM Rho antagonist, or about 1 μM Rho antagonist to about 10 μM Rho antagonist.

In yet other instances, a therapeutically effective amount or dosage of a Rho antagonist can range from about 0.001 mg/kg body weight to about 100 mg/kg body weight, e.g., from about 0.01 mg/kg body weight to about 50 mg/kg body weight, from about 0.025 mg/kg body weight to about 25 mg/kg body weight, from about 0.1 mg/kg body weight to about 20 mg/kg body weight, from about 0.25 mg/kg body weight to about 20 mg/kg body weight, from about 0.5 mg/kg body weight to about 20 mg/kg body weight, from about 0.5 mg/kg body weight to about 10 mg/kg body weight, from about 1 mg/kg body weight to about 10 mg/kg body weight, or about 5 mg/kg body weight.

In other instances, a therapeutically effective amount or dosage of a Rho antagonist can range from about 0.001 mg to about 50 mg total, e.g., from about 0.01 mg to about 40 mg total, from about 0.025 mg to about 30 mg total, from about 0.05 mg to about 20 mg total, from about 0.1 mg to about 10 mg total, or from about 1 mg to about 10 mg total.

A physician will appreciate that certain factors may influence the dosage required to effectively treat a subject, including but not limited to the severity of the disease or disorder, previous treatments, the general health and/or age of the subject, and other diseases present. Moreover, treatment of a subject with a therapeutically effective amount of a Rho antagonist can include a single treatment or a series of treatments. In one example, a subject is treated with a Rho antagonist in the range of between about 0.06 mg to 120 mg, one time per week for between about 1 to 10 weeks, alternatively between 2 to 8 weeks, between about 3 to 7 weeks, or for about 4, 5, or 6 weeks. It will also be appreciated that the effective dosage of a Rho antagonist used for treatment may increase or decrease over the course of a particular treatment.

The pharmaceutical compositions can be included in a container, pack, or dispenser together with instructions for administration.

When a pharmaceutical composition described herein is applied locally, such pharmaceutical composition can include a tissue adhesive and/or fibrin, which can be a tissue or fibrin sealant, e.g., Tisseel®. For example, a pharmaceutical composition can be prepared by mixing a Rho antagonist, e.g., BA-210, with a flowable carrier component, e.g., a tissue adhesive, e.g., a fibrin glue or a collagen gel, capable of forming a therapeutically acceptable matrix in vivo.

Fibrin sealants have three basic components: fibrinogen concentrate, calcium chloride and thrombin (see, e.g., U.S. Pat. No. 7,141,428). Other components can be added to affect the properties of the gel formation, e.g., to modulate the time it takes for the fibrin gel to form from the soluble components, to affect the size of the protein network that is formed, and to affect the strength of the gel. Protease inhibitors can be added to slow down the removal of the gel after it is placed in the body.

Generally, when the components are mixed, a fibrin coagulum is formed in that the fibrinogen molecule is cleaved through the action of thrombin to form fibrin monomers which polymerize spontaneously to form a three-dimensional network of fibrin, largely kept together by hydrogen bonding. This corresponds to the last phase of the natural blood clotting cascade, the coagulation rate being dependent on the concentration of thrombin used. In order to improve the tensile strength, covalent cross-linking between the fibrin chains is provided for by including Factor XI11 in the sealant composition. In the presence of calcium ions, thrombin activates Factor XI11 to Factor XIIIa. Activated Factor XIIIa together with thrombin catalyzes the cross-linkage of fibrin and increases the strength of the clot. The strength of the fibrin clot is further improved by the addition of fibronectin to the composition, the fibronectin being crosslinked and bound to the fibrin network formed. During wound healing the clot material undergoes gradual lysis and is completely absorbed. To prevent too early of degradation of the fibrin clot by fibrinolysis, the fibrin sealant composition can include a plasminogen activator inhibitor or a plasmin inhibitor, such as aprotinin. Such an inhibitor can also reduce the fibrinolytic activity resulting from any residual plasminogen in the fibrinogen composition. Similarly, compositions can include hyaluronic acid (or other polysaccharides), and these can also include a hyaluronidase inhibitor, such as one or more flavonoids (or corresponding inhibitors for other polysaccharides) in order to prevent degradation (i.e., to prolong the duration) of the hyaluronic acid component by hyaluronidase, which is always present in the surrounding tissues. The hyaluronic acid can be crosslinked, a commercially available example being Hylan® (available from Biomatrix, Ritchfield, N.Y., USA). The hyaluronic acid composition can be, e.g., a gel or a solution.

In some instances, fibrin clots can be used for the administration of a Rho antagonist. For example, by incorporating a Rho antagonist described herein into a tissue adhesive, the antagonist is enclosed in a fibrin network formed upon application of the tissue adhesive. This ensures that the antagonist is kept at the site of application while being controllably released from the fibrin network.

The use of fibrin sealants is known in the art (see, e.g., U.S. Pat. No. 5,714,370; U.S. Pat. No. 5,750,657; U.S. Pat. No. 5,773,418; U.S. Pat. No. 5,962,405; U.S. Pat. No. 5,962,420; U.S. Pat. No. 6,117,425; U.S. Pat. No. 6,162,241; and U.S. Pat. No. 6,262,236). Generally, to make a fibrin gel, soluble thrombin and fibrinogen are mixed in the presence of calcium chloride. When the components mix, a fibrin adhesive gel is formed because the fibrinogen molecule is cleaved by thrombin to form fibrin monomers. The fibrin monomers polymerize spontaneously to form a three dimensional network of fibrin, a reaction that mimics the final common pathway of the clotting cascade, i.e., the conversion of fibrinogen to fibrin sealant. For preparations, the fibrinogen and thrombin components should be kept separate until use, so that the polymerization can be controlled with the desired timing before or after application to the subject.

One exemplary fibrin sealant used to formulate/deliver a pharmaceutical composition described herein is TISSEEL VH, Two-Component Fibrin Sealant, Vapor Heated, Kit (TISSEEL VH Fibrin Sealant) manufactured by Baxter Healthcare Corporation. The TISSEEL VH Fibrin Sealant kit contains Fibrinogen (Sealer Protein Concentrate) and Thrombin as the main active ingredients. It also contains Calcium Chloride Solution, and Fibrinolysis Inhibitor Solution (Aprotinin, bovine). The two reconstituted components, the Sealer Protein Solution and Thrombin Solution, are mixed and applied topically. Mixing the Sealer Protein Solution and Thrombin Solution produces a viscous solution that quickly sets into an elastic coagulum. Thrombin is a highly specific protease that transforms the fibrinogen contained in Sealer Protein Concentrate into fibrin. Most of the thrombin is adsorbed by the fibrin so formed. Excess thrombin, if any, is inactivated by protease inhibitors in the blood. Fibrinolysis Inhibitor Solution (Aprotinin) is a polyvalent protease inhibitor which prevents premature degradation of fibrin.

Other fibrin sealants that can be used to formulate/deliver a pharmaceutical composition described herein include, e.g., Cebus™, Ateles™ and Proleus™ (PlasmaSeal); Vivostat® (Vivolution); CryosSeal FS® (Thermogenesis); CoSeal™ (Angiotech); Duraseal® (Confluent Surgical); Poliphase® (Avalon Medical); Bioglue® (Cryolife Inc.); Avitene Fluor™ (Davol); Dermabond™ (Johnson & Johnson); Hemassel, Hemassel-HMN and Hemaseel-Thrombin (Hermacure); Beriplast-P® (Aventis); Fibrocaps® (Profibrix); and Crosseal™, Evicel™ and Thrombin (Omrix Pharmaceuticals).

Still other fibrin sealants that can also be used in the methods and compositions described herein are those described in U.S. RE39,298; U.S. RE39,321; U.S. Pat. No. 4,427,650; U.S. Pat. No. 4,427,651; U.S. Pat. No. 4,414,976; U.S. Pat. No. 4,640,834; U.S. Pat. No. 5,290,552; U.S. Pat. No. 5,607,694; U.S. Pat. No. 5,714,370; U.S. Pat. No. 5,750,657; U.S. Pat. No. 5,773,418; U.S. Pat. No. 5,962,405; U.S. Pat. No. 5,962,420; U.S. Pat. No. 6,117,425; U.S. Pat. No. 6,162,241; U.S. Pat. No. 6,262,236; U.S. Pat. No. 6,780,411; in U.S. application Ser. No. 11/112,156; and in EP Patent No. 0 804 257. Tissue adhesive formulations are also described in U.S. Pat. No. 7,141,428.

It should be understood that these fibrin sealants are named by way of example and are not meant to be limiting. It is to be understood that any pharmaceutically acceptable tissue adhesive such as a fibrin or collagen gel can be used in the methods described herein.

A person of ordinary skill in the art will appreciate that the pharmaceutical compositions described herein can be formulated as single-dose vials. For example, single-dose vials can be produced containing about 25 μg, about 40 μg, about 60 μg, about 100 μg, about 150 μg, about 200 μg, about 300 μg, or about 500 μg of a Rho antagonist-containing pharmaceutical composition described herein. In a further example, single-dose vials can be produced containing a concentration of about 0.5 mM or about 1.0 mM of a pharmaceutical composition described herein.

Treatment of a subject with a therapeutically effective amount of a Rho antagonist-containing pharmaceutical composition described herein can be a single treatment, continuous treatment, or a series of treatments divided into multiple doses. The treatment can include a single administration, continuous administration, or periodic administration over one or more years. Chronic, long-term administration can be indicated in many cases. In some instances, a subject is treated for up to one year. In other instances, a subject is treated for up to 6 months. In yet another situation, a subject is treated for up to 100 days. In one example, a subject is treated with a Rho antagonist in a time frame of one time per week for between about 1 to 10 weeks, alternatively between 2 to 8 weeks, between about 3 to 7 weeks, or for about 4, 5, or 6 weeks. In other instances, a subject can be treated substantially continuously. In other situations, a subject can be treated once per day, twice per day, once per week, or once per month.

Generally, each formulation is administered in an amount sufficient to suppress or reduce or eliminate a deleterious effect or a symptom of a bone disorder or condition described herein.

In addition to treating pre-existing bone disorders, the methods described herein can prevent or slow the onset of such disorders. For example, the Rho antagonists described herein can be administered for prophylactic applications, e.g., can be administered to a subject susceptible to or otherwise at risk for a bone disorder. In some instances, a Rho antagonist can be administered to a subject who has a pre-existing bone disorder and is susceptible to or otherwise at risk for a further bone disorder.

Suppression of a bone disorder can be evaluated by any known methods of measuring whether bone resorption is slowed or diminished. Such methods include, e.g., direct observation and indirect evaluation, e.g., by evaluating subjective symptoms or objective physiological indicators, or bone imaging procedures, or bone biopsy, or serum markers, e.g., phosphate, calcium, tartrate-resistant acid phosphatase (TRACP), alkaline phosphatase (AlP), intact osteocalcin (OC), or the carboxyterminal pyridinoline cross-linked telopeptide of type I collagen (ICTP). Treatment efficacy can be evaluated, e.g., based on the prevention of bone loss or restoration of bone, as assessed by the individual indictors given above or any combination thereof.

In other instances, a pharmaceutical composition described herein is used as a drug-eluting coating for a medical device including, but not limited to, e.g., a stent, cage, bone, screw, rod, post, plate, dental implant, mesh, wire, nail, anchor, artificial joint, sponge, polymer, or gel.

### Combination Therapy

In some instances, a Rho antagonist described herein is administered in combination with one or more therapeutic agents, e.g., therapeutic agents useful in the treatment of bone disorders or conditions described herein. For example, certain second therapeutic agents can promote tissue growth or infiltration, such as growth factors. Exemplary growth factors for this purpose include, without limitation, epidermal growth factor (EGF), fibroblast growth factor (FGF), platelet-derived growth factor (PDGF), transforming growth factors (TGFs), parathyroid hormone (PTH), leukemia inhibitory factor (LIF), and insulin-like growth factors (IGFs). Other second therapeutic agents can promote bone growth, such as bone morphogenetic proteins (U.S. Pat. No. 4,761,471; PCT Pub. WO 90/11366), osteogenin (Sampath, et al., Proc. Natl. Acad. Sci. USA (1987) 84:7109 13) and NaF (Tencer, et al., J. Biomed. Mat. Res. (1989) 23: 571 89).

In other instances, a Rho antagonist described herein can be used in combination with agents that inhibit bone resorption, such as antiresorptive agents including but not limited to, e.g., estrogen, bisphosphonates, calcitonin, PTH, and BMPs.

In some instances, a Rho antagonist is administered in combination with one or more Rho kinase (ROCK) inhibitors. ROCK inhibitors are known in the art and include, without limitation, Fasudil, Y27632, WF-536, SC-3536, H-89, ML-9, HA-1077, and H-1152 (see, e.g., Somiyo, Nature 389:908-910, 1997; Uehata et al., Nature 389:990-994, 1997; U.S. Pat. No. 4,997,834). Other ROCK inhibitors are described in US Pub. Nos. 2004/0138272 and 2005/0272751.

The invention is further illustrated by the following examples. The examples are provided for illustrative purposes only. They are not to be construed as limiting the scope or content of the invention in any way.

### EXAMPLES

### Example 1

### Calvarial Organ Culture (Bone Resorption and DNA Synthesis)

### Protocol

Twenty mice are injected subcutaneously at 1-3 days old with 3 μCi ⁴⁵Ca in a 25 μl sterile saline solution. 3 ml of culture medium (0.1% BSA/Media 199) are added to a 30 mm Petri dish and incubated at 37° C. Mice are decapitated and the hemi-calvariae are dissected from the heads and placed on grids (surface tension pulls media up under the bone). Bones are pre-cultured for 24 hours. The medium is replaced with 3 ml fresh medium, and 30 μl/well Cethrin® is added and the bones incubated for 48 hr.

300 μl media from each dish is placed into a 5 ml plastic vial, to which 3-4 ml scintillant is added and vortexed. The vials are counted for ⁴⁵Ca in a beta counter. 2 μCi of ³H-thymidine in 500 μl media is added to all dishes and incubated for 4 hr. Sample bones are rinsed in fresh media and then placed in separate glass tubes containing 1 ml of 5% trichloroacetic acid (TCA). Bones are incubated at 4° C. for 12-24 hr. 100 μl TCA solution is removed from each tube and placed in a 5 ml plastic vial to which 3-4 ml scintillant is added and vortexed. The ⁴⁵Ca in the vials is counted in a beta counter.

The amount of bone resorption is measured by calculating the percentage of ⁴⁵Ca released from bone using the following equations:

$$\begin{matrix}\% {}^{45} Ca release = \frac{ \begin{matrix}no . of {}^{45} Ca disintegrations \text{/} min . \\ in culture media\end{matrix} }{total no . of {}^{45} Ca disintegrations \text{/} min .} \times 100 & ( 1 ) \\ total no . {}^{45} Ca disintegrations \text{/} min . = no . of {}^{45} Ca disintegrations in the media + no . of {}^{45} Ca disintegrations \text{/} min . in TCA soln . & ( 2 )\end{matrix}$$

To measure osteoclast activity, the amount of DNA synthesis within the bone samples are measured. TCA aliquot is removed, TCA is tipped off, 1 ml of acetone is added, and the sample left in a fume hood at room temperature (RT) for 1-2 hr. Acetone is removed, and 1 ml of ether is added for 1-2 hr. The ether is removed, and the bones are allowed to dry thoroughly. Bones are then weighed (μg). Bones are placed in 20 ml glass scintillation vials, and 500 μl of 1 M KOH is added. The vials are tightly capped and incubated at 80° C. for about 10 min. 500 μl of 1 M HCl is added to all vials to neutralize the solution. 10 ml of scintillant is added to each vial, vortexed thoroughly, and the amount of ³H is counted. The amount of DNA synthesis is calculated using the following equation:

$$DNA synthesis ( disintegrations \text{/} min . \text{/} µg ) = \frac{ \begin{matrix}no . {}^{3} H disintegrations / min . \\ of dissolved bone\end{matrix} }{bone weight}$$

### Results

The treatment of bone with Cethrin® results in a reduction in bone resorption relative to bones not treated with Cethrin®. This reduction in bone resorption is due to a reduction in the level of osteoclast activity.

### Example 2

### Mineralization of MC3T3-E1 Cells

### Protocol

MC3T3-E1 cells are maintained in culture medium (MEM+10% FCS+Sodium Pyruvate (1 ml stock/100 ml media). When the cells are growing well and are ready to seed, tyrosinase is added to them and the cells are washed using a standard procedure. The cells are then counted and seeded at a density of 5×10⁴ cells/well in a 6-well plate or 35 mm dish. Cells are allowed to grow until confluent (about 3 days) in culture medium, and then the medium is replaced with the mineralization medium (AMEM, 10% FCS, 50 μg/ml ascorbic-2-phosphate, and 10 mM P-glycerophosphate) and Cethrin® is added. Media and Cethrin® are replaced twice weekly. The cells are then stained with Von Kossa or Alizarin Red after 18-21 days of mineralization.

### Results

The use of Cethrin® within the mineralization media results in increased mineralization relative to the absence of Cethrin®, as determined by new bone staining with Von Kossa or Alizarin Red.

### Example 3

### Osteoblast Proliferation Assay

### Protocol

The overall protocol is as follows: On day 1, primary fetal rat calvarial osteoblasts were seeded in MEM/5% FCS. On day 2, cells were serum-starved with MEM/0.1% BSA. On day 3, fresh MEM/0.1% BSA and Cethrin were added. On day 4, ³H thymidine was added and the cells were incubated for 6 hours. Plates were then washed and TCA was added. On day 5, plates were processed for counting.

24-well plates were seeded as follows: Flasks were rinsed 2× with 4 ml of PBS. 4 ml of trypsin were added and the flasks were placed in an incubator for approximately 5 minutes. When cells had lifted off, 8 ml of MEM+5% FBS were added into a 15 ml centrifuge tube and the tube was spun at 1200 rpm for 2 minutes. Supernatant was discarded and the cells were washed with 5 ml of media. The cells were then re-spun and resuspended in 5 ml of media.

Cell counts were performed using a 1:20 dilution (i.e., 50 μl cells+950 μl media in Eppendorf tubes). The number of cells were counted in 8 large squares of haemocytometer and the average was determined. Cells were added to 24 well plates at a density of 5×10⁴ cells/ml MEM+5% FBS, using 0.5 ml per well. Cells were added to 6-well plates at a density of 5×10⁴ cells/ml MEM+5% FBS, using 3 ml per well.

Media was removed using a vacuset, and 1.0 ml of MEM+0.1% BSA was added to each well: (333 μl BSA stock in 100 ml MEM). The cells were incubated at 37° C. overnight.

The following day, [³H] thymidine was added to the wells. A final concentration of 0.5 μCi/50 μl per well (50 μl of stock (5 mCi)+5 ml of media) was used, which was sufficient for 4×24 well plates. 50 μl [³H] thymidine (50 μl of stock+5 ml of media) was added to each well and incubated for 6 hrs at 37° C. The wells were then washed with 1 ml of serum-free media. The media was removed and 1 ml of 10% TCA was added to each well.

TCA was removed and each well was rinsed with 1 ml of ethanol/ether (3:1). The ethanol/ether was then completely evaporated. 200 μl of 2 M KOH was added and the wells were incubated at 55° C. for 30 min. 400 μl of 1 M HCl was then added to each well. 300 μl of each sample was placed into a scintillation vial, scintillation fluid was added and scintillation was performed.

### Results

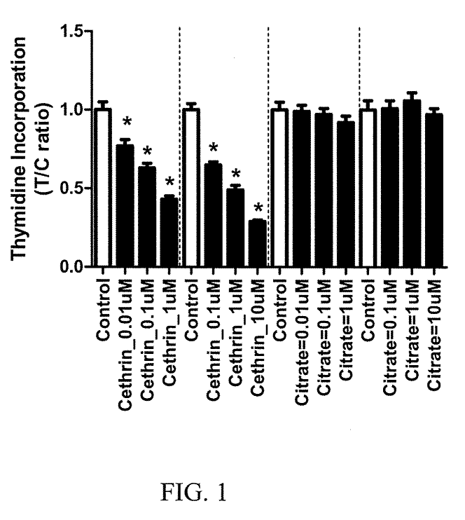

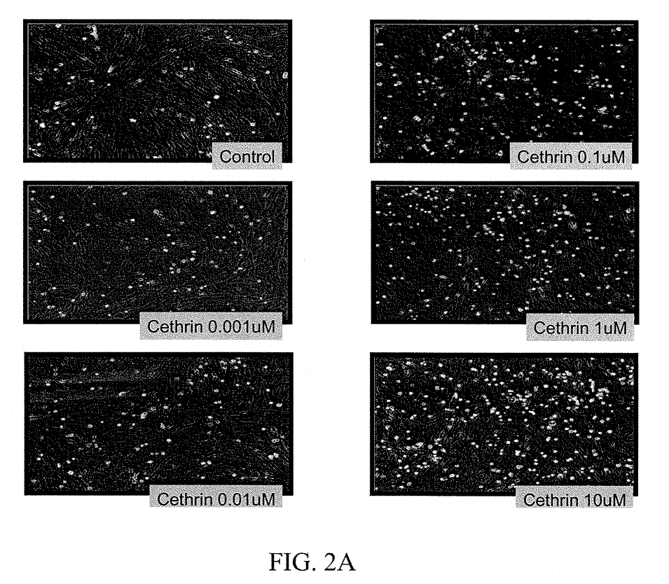

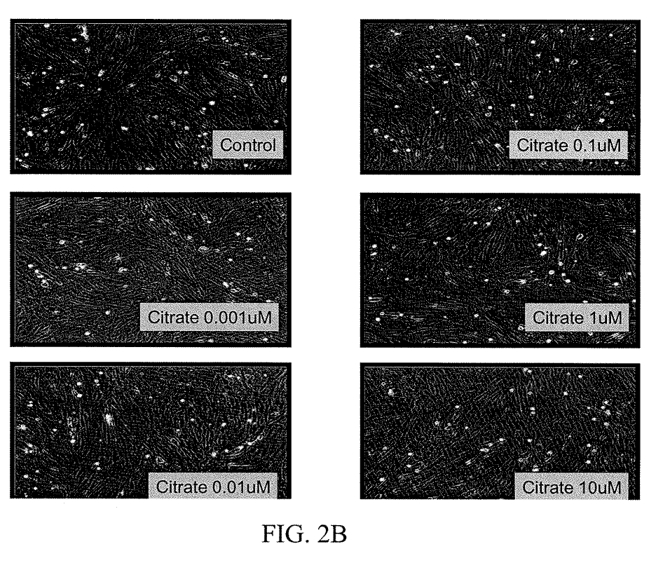

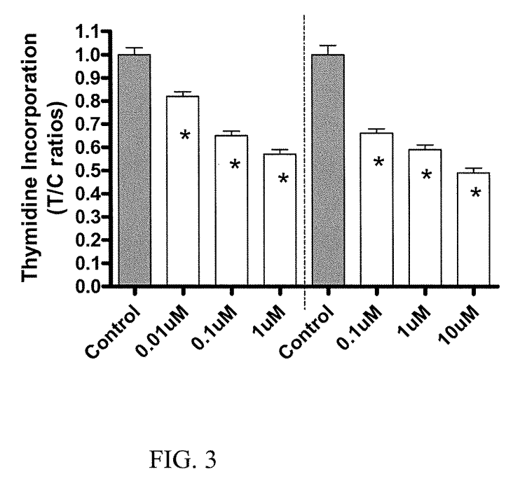

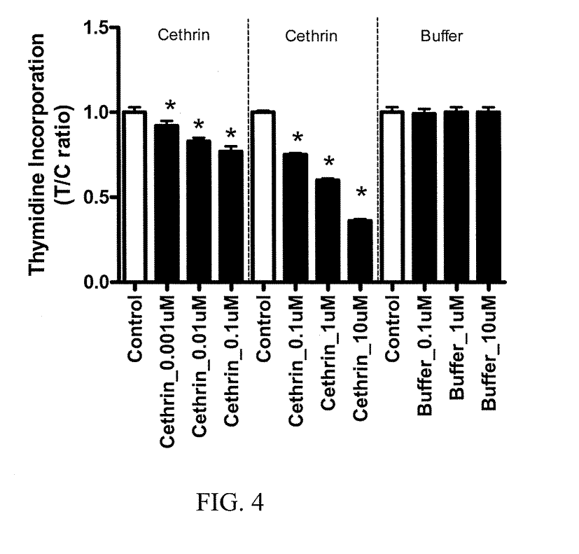

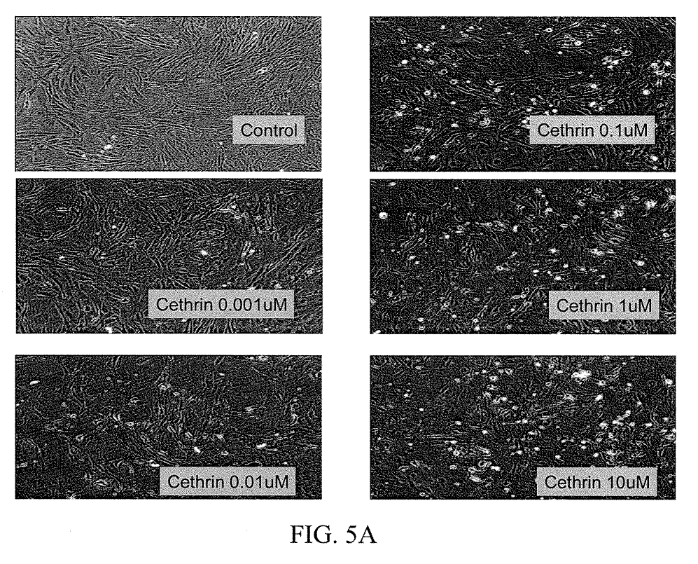

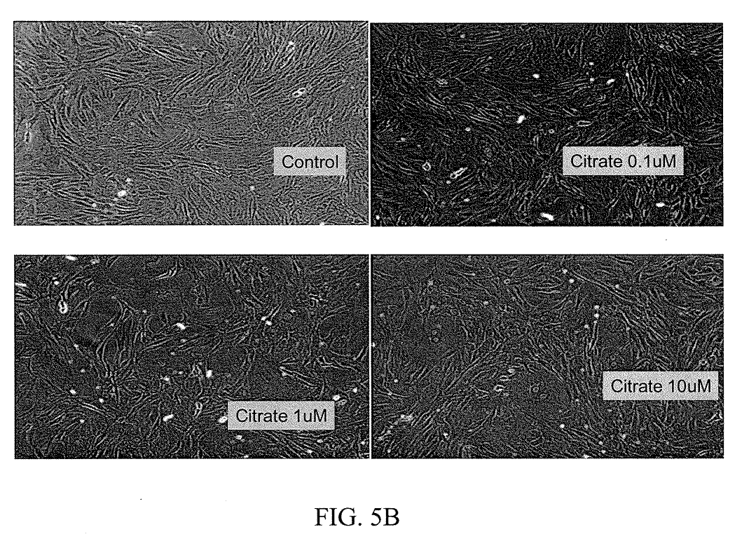

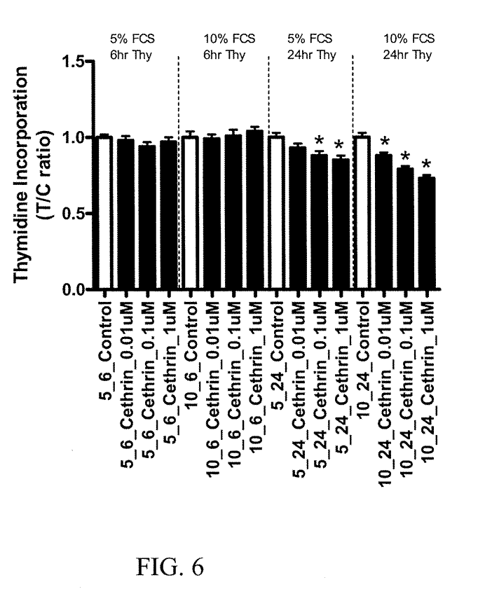

Cethrin® dose-dependently decreased thymidine incorporation compared to control using the above protocol of non-actively growing cells (FIG. 1). The cells appeared to round-up and die in a dose-responsive manner (FIG. 2a), compared to citrate buffer controls, which had no effect on the cells (FIG. 2b). These results were confirmed in the MCT3T-E1 osteoblast cell line, in which Cethrin® also inhibited thymidine incorporation in a dose-dependent manner compared to control (FIG. 3). Similar results were seen in actively growing primary fetal rat calvarial osteoblasts, where the cells were not serum starved but were transferred to media with 1% FCS rather than 0.1% BSA (FIGS. 4, 5a, and 5b; citrate buffer had no effect on actively-growing cells). When 5% or 10% FCS was added to cultures, Cethrin® inhibited thymidine incorporation at 24 hrs (FIG. 6). At 6 hrs, there were no significant changes in thymidine incorporation compared to control (FIG. 6).

Cethrin® dose-responsively decreased osteoblasts and the addition of FCS into the cultures did not rescue this decrease. As Rho is known to be involved in regulating cytoskeletal organization, it is not surprising that the cells appear to be “rounding-up”. In addition, preliminary osteoblast long term cultures grown to the bone nodule/mineralization stage indicate that Cethrin® may be encouraging osteoblast differentiation.

### Example 4

### Mouse BM Culture for Osteoclast Formation

### Protocol

After anesthetic, each mouse was sacrificed by cervical dislocation. The legs were sterilized with ethanol and removed at the hip joint. The feet were cut off above the ankle and rinsed in 100% ethanol. The skin was removed by peeling off from the hip end to the ankle joint. In a separate dish using a scalpel and tweezers, muscle was scraped away to expose the length of the bone (femur or tibia). The epiphyses were cut off from each end of the femur and tibia providing a large enough diameter to insert a 23 GA needle. The bone was placed in αMEM. Using a 23 GA needle and a 5 ml syringe filled with αMEM, each bone was flushed through both ends to remove bone marrow. Media plus marrow was collected in a 50 ml conical tube. Using the αMEM and marrow collected from the bone marrow dissection, the tube was spun at 1200 rpm for 2 mins. The media was poured off and the pellet was re-suspended in approximately 15 ml of 10% FCS/αMEM.

7.5 ml of cell suspension was plated in two 9 cm Petri dishes and incubated at 37° C., 5% CO₂ for two hrs. After two hours, the media and non-adherent cells from both plates were removed and placed in a 15 ml centrifuge tube. The tube was spun at 1200 rpm for 2 mins. The media was gently poured off and approximately 4-5 ml of 10% FCS/aMEM was added and the media was aspirated. To 50 μL of each cell suspension was added 950 μL of media in an Eppendorf tube. Cells were counted in a haemocytometer chamber. (Cell counting: average cell number×dilution×10⁴ cells/ml). 5×10⁵ cells in 500 μL medium were plated into each well. 5 μL of 10⁻⁶ M Vit D₃ were added to each well except to “no vitamin” control wells. Cethrin® was added as indicated to appropriate wells and each plate was incubated at 37° C., 5% CO₂ for 48 hrs.

On Day 2, 500 μL of 10% FCS/αMEM was added to “no vitamin” control wells. To all other wells, 500 μL of 15% FCS/αMEM containing Vit D₃ was added. 10 μL of drug treatment was added to each appropriate well and then incubated at 37° C., 5% CO₂ for 48 hrs. On Day 4, 500 μL was removed by reverse pipetting from each well followed by incubation at 37° C., 5% CO₂ for 72 hrs. On Day 7, the wells were stained for TRAPi cells.

### Results

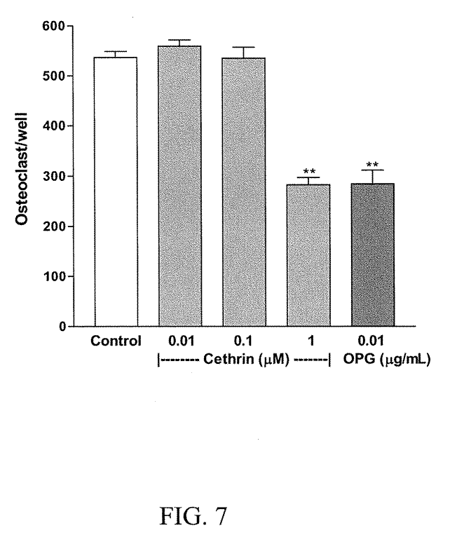

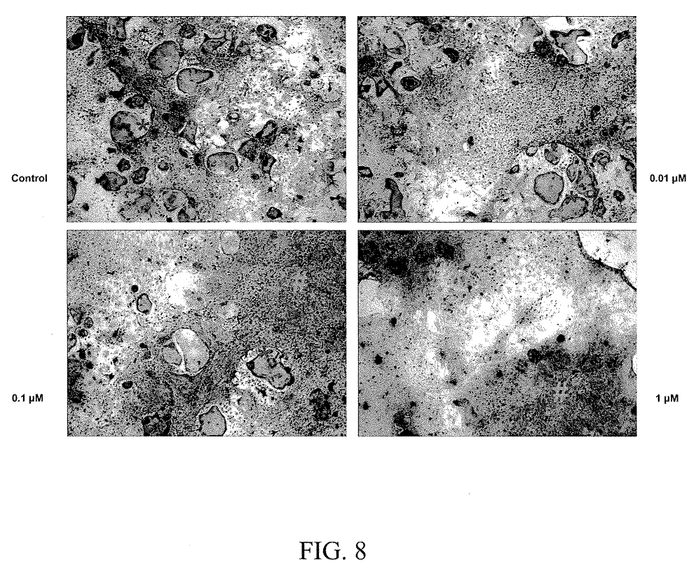

Cethrin® decreased osteoclast development (FIG. 7). The stromal cells (in comparison to the osteoblast cultures above) were not inhibited in their growth, as there were healthy numbers of stromal cells surrounding the TRAP-positive multinucleated osteoclasts as indicated in the photomicrographs (FIG. 8; “*” indicates osteoclasts (multinucleated cells); “#” indicates stromal cells (including osteoblasts)).

Cethrin® specifically inhibits osteoclastogenesis, as the stromal cells are not inhibited.

### Example 5

### Cethrin® Rabbit Radial Defect Study

### Protocol

The objective of this experiment is to evaluate the ability of Cethrin® applied locally in a fibrin sealant matrix to the site of surgically-created bone deficit to stimulate healing of critical-length defects in long bones. The test compares healing in Cethrin® treated bones with healing in autografted bones, and develops a dose-response curve to predict the most efficacious Cethrin® dose.

The test system used for evaluation is the reduction of critical length segmental defect in the radius of skeletally mature female New Zealand white rabbits.

30 skeletally mature (≧26 week-old, documented) female New Zealand white rabbits are randomly assigned to five groups (N=6/group) and receive the following treatments: fibrin matrix vehicle control (Group1), 1 μg BA-210 in fibrin matrix (Group 2), 10 μg BA-210 in fibrin matrix (Group 3), 100 μg BA-210 in fibrin matrix (Group 4), and morselized iliac crest bone graft (ICBG) as positive control (Group 5).

TABLE 1
Experimental Design

| Treatment | | | Sacrifice |
|---|---|---|---|
| group | n | Treatment | (week) |
| 1 | 6 | Neg. Control - Fibrin Matrix | 8 |
| 2 | 6 | Cethrin ® (1 μg BA-210 per defect) | 8 |
| 3 | 6 | Cethrin ® (10 μg BA-210 per defect) | 8 |
| 4 | 6 | Cethrin ® (100 μg BA-210 per defect) | 8 |
| 5 | 6 | Positive control - morselized Iliac Crest | 8 |
| | | Bone Graft | |

On the day of surgery, rabbits are anesthetized and posteroanterior and lateral radiographs are taken of both forelimbs. The left forelimb is shaved, prepared for surgery with alternating scrubs of povidone iodine and alcohol, and sterilely draped. Prophylactic antibiotics (cefazolin, 20 mg/kg) are administered preoperatively, as are fentanyl transdermal patches (25 μg/hr). A skin incision of approximately 3 cm is made over the craniomedial surface of the radius, and the underlying tissues are separated and retracted. Under irrigation, an oscillating saw is used to make two transverse cuts through the radius, yielding a 1.5 cm segmental defect with its distal margin located 2.5 cm from the radiocarpal joint. Once the defect is created the assigned treatment material is placed in the bony defect and the subcutaneous soft tissues and skin are closed with Vicryl suture (Ethicon, Inc., Somerville, N.J.). Post-operative pain is managed with fentanyl transdermal patches (25 μg/hr) and once-daily injections of carprofen (4 mg/kg) for three days. For the animals treated with RhoA inhibitor, BA-210 solution (25 μL) is delivered in 600 μL of fibrin clot (300 μL fibrinogen and 300 μL thrombin). Aliquots of BA-210 (1 μg, 10 μg, and 100 μg) are prepared from a 30 mg/ml stock solution via dilution with citrate buffer at the time of surgery. The negative control animals (Group 1) receive 600 μL of fibrin alone (with 25 μl of citrate buffer), while the positive controls (Group 5) receive 0.3 cm³ of morselized corticocancellous autograft from the iliac crest bone.

For the positive control animals, graft bone is harvested from the iliac crest prior to the segmental defect surgery. In these animals the caudal back area is shaved and prepared for surgery with alternating scrubs of povidone iodine and alcohol, and sterilely draped. A 4-6 cm incision is made on the midline and the subcutaneous tissues overlying the cranial portions of one iliac crest are approached via blunt, sharp and electrocautery dissection. Muscular attachments on the iliac crests are removed via subperiosteal elevation and corticocancellous bone is harvested and morselized with a rongeur. The deep and subcutaneous tissues are closed with 4-0 Vicryl, and the skin is closed with staples.

Plane X-rays of the forelimb are taken at two-week intervals to monitor healing and to detect new bone formation. All of the animals are euthanized eight weeks post-operatively. At euthanasia, posteroanterior and lateral radiographs are taken of the forelimbs in situ for comparison with the preoperative radiographs. The radii and ulnae are then harvested en bloc, and placed in cold fixative (10% formalin). The proximal and distal ends of the bones are trimmed, and high-resolution 3-D volume images (30 μm³ isometric voxel size) are generated of the defect sites using a microcomputed tomography system (μCT40, Scanco Medical AG, Scanco Corporation, CH). The scans are performed at 55 kVp and 145 μA. Defect bridging and new bone formation are qualitatively scored using the 5 point grading system described by Bodde et al. (J. Biomed. Mater. Res. 85A:206-217 (2008)) and Hedberg et al. (Tissue Engineering 11:1356-1367 (2005)). Defect new bone volume is then quantified from a standardized 500-slice (15 mm) volume centered on the defect site. Ulnar bone is removed from the volume by manual segmentation. Bone volume is calculated using the μCT40's built-in image processing routines, with fixed filtering and segmentation parameters of σ=1.0, support=1 and threshold=315. Mineral density (in mg hydroxyapatite per cubic centimeter, mg HA/cm³) is calculated via density calibration of the segmented images. Statistical comparisons are made using a one-way analysis of variance (ANOVA) with Holm-Sidak post-hoc tests.

After μCT imaging the bones are decalcified and embedded in paraffin, and serial mid-sagittal sections are cut with a microtome. The sections are mounted on glass slides and alternating serial sections are stained with toluidine blue and Mason's Trichrome for routine light microscopic analysis.

### Results

Local application of Cethrin® in a fibrin sealant matrix accelerates the formation of new bone at the site of bone deficit when compared to the application of fibrin sealant alone.

### Example 6

### Administration of Cethrin® and BMP to Rabbit Radial Defect

In another study, rabbits are prepared and treated as described in Example 5. However, Cethrin® and BMP are administered at the same time. Cethrin® is administered locally in fibrin sealant, and BMP is administered either locally with Cethrin® or systemically.

The combined administration of BMP and Cethrin® results in a synergistic or additive effect on the acceleration the formation of new bone at the site of bone deficit when compared to the application of Cethrin® in fibrin sealant alone.

### Example 7

### Ex Vivo Treatment of Osteoblast Precursor Cells

Osteoclast precursor cells are identified by a marker, such as osteopontin, and are isolated from a subject using routine methods. The osteoclast precursor cells are maintained ex vivo and treated with Cethrin®. The treated cells are then implanted into the subject at the site of bone injury.

The ex vivo treatment of osteoblast precursor cells with Cethrin® encourages the formation and maturation of osteoblasts so as to promote bone formation when implanted into the subject.

### EQUIVALENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method of modulating Rho GTPase in a bone cell, comprising contacting the bone cell with an amount of a Rho antagonist that modulates Rho GTPase activity.

2. The method of claim 1, wherein the Rho antagonist is a C3 polypeptide, a C3-like polypeptide, or a biologically active fragment thereof.

3. The method of claim 1, wherein the Rho antagonist is a conjugate comprising a C3 polypeptide, a C3-like polypeptide, or a biologically active fragment thereof.

4. The method of claim 3, wherein the conjugate is a fusion protein comprising a C3 polypeptide, a C3-like polypeptide, or a biologically active fragment thereof.

5. The method of claim 4, wherein the conjugate comprises the amino acid sequence of SEQ ID NO:1.

6. The method of claim 1, wherein the bone cell is a bone remodeling cell.

7. The method of claim 6, wherein the bone remodeling cell is an osteoblast or an osteoblast precursor cell.

8. The method of claim 6, wherein the bone remodeling cell is an osteoclast or an osteoclast precursor cell.

9. A method of promoting bone remodeling in a subject, comprising administering to the subject a therapeutically effective amount of a Rho antagonist, thereby promoting bone remodeling in the subject.

10. The method of claim 9, wherein the Rho antagonist is a C3 polypeptide, a C3-like polypeptide, or a biologically active fragment thereof.

11. The method of claim 9, wherein the Rho antagonist is a conjugate comprising a C3 polypeptide, a C3-like polypeptide, or a biologically active fragment thereof.

12. The method of claim 11, wherein the conjugate is a fusion protein comprising a C3 polypeptide, a C3-like polypeptide, or a biologically active fragment thereof.

13. The method of claim 12, wherein the conjugate comprises the amino acid sequence of SEQ ID NO:1.

14. A method of treating a bone injury in a subject, comprising administering to the subject a therapeutically effect amount of a Rho antagonist, thereby treating the bone injury.

15. The method of claim 14, wherein the bone injury is a bone fracture.

16. The method of claim 14, wherein the bone injury is a spinal injury.

17. The method of claim 14, wherein the bone injury is bone loss incident to a metastasizing tumor.

18. The method of claim 14, wherein the Rho antagonist is administered locally to the site of the bone injury.

19. The method of claim 18, wherein the Rho antagonist comprises the amino acid sequence of SEQ ID NO:1.

20. The method of claim 19, wherein the Rho antagonist is administered at a dosage of from about 0.0001 μg/cm³ tissue to about 100 μg/cm³ tissue.

21. The method of claim 18, wherein the Rho antagonist is administered in a fibrin sealant.

22. The method of claim 21, wherein the fibrin sealant is TISSEEL®.

23. The method of claim 18, wherein the Rho antagonist is administered directly to bone tissue using a drug eluting device.

24. A method of stimulating an osteoblast precursor cell to differentiate into an osteoblast, comprising contacting the osteoblast precursor cell with a Rho antagonist, thereby stimulating the differentiation of the precursor cell.

25. The method of claim 24, wherein the Rho antagonist is a C3 polypeptide, a C3-like polypeptide, or a biologically active fragment thereof.

26. The method of claim 24, wherein the Rho antagonist is a conjugate comprising a C3 polypeptide, a C3-like polypeptide, or a biologically active fragment thereof.

27. The method of claim 26, wherein the conjugate is a fusion protein comprising a C3 polypeptide, a C3-like polypeptide, or a biologically active fragment thereof.

28. The method of claim 27, wherein the conjugate comprises the amino acid sequence of SEQ ID NO:1.

29. A method of inhibiting bone resorption in a subject, comprising administering to the subject a Rho antagonist, thereby inhibiting bone resorption.

30. The method of claim 29, wherein the Rho antagonist is a C3 polypeptide, a C3-like polypeptide, or a biologically active fragment thereof.

31. The method of claim 29, wherein the Rho antagonist is a conjugate comprising a C3 polypeptide, a C3-like polypeptide, or a biologically active fragment thereof.

32. The method of claim 31, wherein the conjugate is a fusion protein comprising a C3 polypeptide, a C3-like polypeptide, or a biologically active fragment thereof.

33. The method of claim 32, wherein the conjugate comprises the amino acid sequence of SEQ ID NO:1.

34. A method of treating a bone injury in a subject, comprising:
obtaining osteoblast precursor cells from the subject;
contacting the precursor cells with a Rho antagonist, thereby stimulating the cells to differentiate into osteoblasts; and
implanting the osteoblasts into the subject.

35. The method of claim 34, wherein the Rho antagonist is a C3 polypeptide, a C3-like polypeptide, or a biologically active fragment thereof.

36. The method of claim 34, wherein the Rho antagonist is a conjugate comprising a C3 polypeptide, a C3-like polypeptide, or a biologically active fragment thereof.

37. The method of claim 36, wherein the conjugate is a fusion protein comprising a C3 polypeptide, a C3-like polypeptide, or a biologically active fragment thereof.

38. The method of claim 37, wherein the conjugate comprises the amino acid sequence of SEQ ID NO:1.
